(19) United States
(12) Patent Application Publication
(10) Pub. No.: US 2002/0115663 A1
(43) Pub. Date: Aug. 22, 2002

(54) Novel compounds

(76) Inventors: Emma Barker, Herne Bay (GB); Styrbjorn Bystrom, Taby (SE); Eric Desarbre, Mulhouse (FR); James Duffy, Herne Bay (GB); Charlotta Liljebris, Knivsta (SE)

Correspondence Address: JANIS K. FRASER, Ph.D., Fish & Richardson P.C., 225 Franklin Street, Boston, MA, 02110-2804, US

(21) Appl. No.: 09/963,733

(22) Filed: Sep. 26, 2001

Related U.S. Application Data
(60) Provisional application No. 60/239,327, filed on Oct. 10, 2000.

(30) Foreign Application Priority Data
Sep. 26, 2000 (SE) 0003436-3
Jun. 1, 2001 (SE) 0101933-0

(51) Int. Cl. C07D 417/14; C07D 495/02; A61K 31/541; A61K 31/5377; A61K 31/5025

(52) U.S. Cl. 514/228.5; 514/248; 514/234.5; 544/60; 544/117; 544/236

(57) ABSTRACT

The present invention is directed to novel pyridazine compounds of the formula I

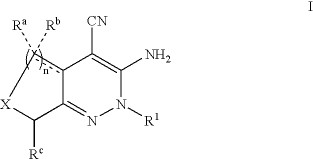

as well as pharmaceutically and pharmacologically acceptable salts, and hydrates thereof; to a process for their preparation, their use and pharmaceutical compositions comprising said novel compounds. These novel compounds are useful in therapy, particularly for the treatment of type 2 diabetes mellitus.

### CROSS REFERENCE TO RELATED APPLICATIONS

This application claims priority from Swedish Patent Application No. 0003436-3, filed Sep. 26, 2000, U.S. Provisional Patent Application Serial No. 60/239,327, filed Oct. 10, 2000, and Swedish Patent Application No. 0101933-0, filed Jun. 1, 2001. These applications are incorporated herein by reference in their entirety.

### FIELD OF THE INVENTION

The present invention is directed to novel compounds, to a process for their preparation, their use and pharmaceutical compositions comprising said novel compounds. These novel compounds are useful in therapy, particularly for the treatment of type 2 diabetes.

### BACKGROUND

Phosphorylation on serine, threonine and tyrosine amino acid residues in downstream proteins forms the major output from growth factor and cytokine receptors, from which a cellular response is built. A large number of growth factor and cytokine-regulated protein tyrosine kinases (PTKs) have been identified which can be integral parts of receptor proteins or cytosolic molecules (Al-Obeidi, F A, Wu, J J & Lam, K S, Biopolym. Pept. Sci. Sect. 47, 197-223). These serve to phosphorylate proteins on tyrosine residues within specific primary amino acid sequences which, when phosphorylated, act as docking points for proteins which contain SH₂ domains. It is the docking of proteins to phosphorylated tyrosine residues which contributes to the activation of such proteins and the establishment of a signal transduction cascade.

The overall output from signal transduction cascades is derived from the balance between phosphorylation and dephosphorylation of proteins. Phosphotyrosines are returned to their free acid form by the action of protein tyrosine phosphatases (PTPs) (Zhang, Z Y (1998) Crit. Rev. Biochem. Mol. Biol., 33, 1-52). Whilst a large number of PTKs has been identified (Hunter, T (1994) Sem. Cell Biol. 5, 367-376), the number of PTPs identified to date is decidedly smaller (van Huijsduijnen, R H (1998) Gene 225, 1-8). Despite the smaller number of enzymes in the PTP family available for investigation, a detailed understanding of the roles they play in signal transduction and disease has not been forthcoming. This is due in part to the lack of small molecule inhibitor molecules which are specific for members of the PTP family and which are permeable to the cell membrane and can thus be used in cell-based experiments. Furthermore, whilst experiments in transgenic animals can be and have been performed in which individual PTPs can be ablated, the effects of the loss of function of a specific enzyme may be masked by compensation by other members of the PTP family. Thus, the availability of small molecule inhibitors of PTPs would be very useful to the study of this important family of enzymes.

A role for the PTP family of proteins in ontogeny and disease is now becoming clearer (Li, L & Dixon, J E (2000) Sem. Immunol. 12, 75-84). Thus, experiments with gene knockouts in transgenic animals has revealed that the motheaten phenotype of mice in which cells of the haematopoietic lineage undergo hyper-proliferation is due to the loss of normal SHPTP1 function (Schultz, L D, Schweitzer, P A, Rajan, T V, Yi, T & Ihle, J N (1993) Cell 73, 1445-1454). Loss of function in the receptor-like subfamily of PTPs leads to conditions such as heightened and reduced sensitivity to insulin (Ren, J-M, Li, P-M, Zhang, W-R, Sweet, L J, Cline, G, Shulman, G I, Livingston, J N & Goldstein, B J (1998) Diabetes 47, 493-497), stunted growth and neurological disruption (Elchelby, M, Wagner, J, Kennedy, T E, Lanctot, C, Michaliszyn, E, Itie, A, Drouin, J & Tremblay, M L (1999) Nature Genet. 21, 330-333) and blockages in T cell maturation (Kishihara, K, Penninger, J, Wallaca, V A, Kundig, T M, Kawai, K, Wakeham, A, Timms, E, Pfeffer K, Ohashi, P S & Thomas P L (1993) Cell 74, 143-156).

The recent descriptions of mice in which the PTP PTP1B had been disrupted revealed that loss of function of this enzyme leads to enhanced insulin sensitivity and resistance to the development of obesity, thus revealing a therapeutic need for the development of specific PTP inhibitors (Elchelby, M, Payette, P, Michaliszyn, E, Cromlish, W, Collins, S, Loy, A L, Normandin, D, Cheng, A, Himms.Hagen, J, Chan, C C, Ramachandran, C, Gresser, M J, Tremblay, M L & Kennedy, B P (1999) Science 283, 1544-1548; Klaman, L D, Boss, O, Peroni, O D, Kim, J K, Martino, J L, Zablotny, J M, Moghal, N, Lubkin, M, Kim, Y-B, Sharpe, A H, Stricker-Krongrad, A, Shulman, G I, Neel, B G & Kahn, B B (2000) Mol. Cell. Biol. 20, 5479-5489). The mechanism of insulin action depends critically upon the phosphorylation of tyrosine residues in several proteins in the insulin-signaling cascade. PTPs that dephosphorylate these proteins are important negative regulators of insulin action. Therefore, the use of specific PTP inhibitors may therapeutically enhance insulin action.

The anabolic effects of insulin are triggered through the activation of a variety of signal transduction cascades which lie downstream of the insulin receptor (Gustafson, T. A., Moodie, S A & Lavan, B E (1999) Rev. Physiol. Biochem. Pharmacol. 137, 71-190). The varieties of signals that are activated by insulin are thought to contribute to the range of effects that insulin controls. However, each pathway is activated by a common series of biochemical reactions proximal to the insulin receptor. Thus, the insulin receptor undergoes autophosphorylation on tyrosine residues when activated by insulin, and also phosphorylates other proteins, in particular, the insulin receptor substrate proteins (IRSs). It has now become widely accepted that the resistance to insulin that is a feature of type 2 diabetes results in part from dysfunctions in signal transduction activated by the insulin receptor, in particular in steps early in the signaling cascades which are common to different pathways (Virkamaki, A, Ueki, K & Kahn, R C (1999) J. Clin. Invest. 103, 931-943; Kellerer, M, Lammers, R & Haring, H-U (1999) Exp. Clin. Endocrinol. Diabetes 107, 97-106).

The signals which emanate from the insulin receptor are switched off by the returning of the insulin receptor and other components of the signal transduction cascades to their basal, non-active states. For the insulin receptor and the IRS proteins, this is achieved by dephosphorylation of phosphotyrosine residues. It is now becoming clear that different PTPs may regulate the insulin receptor in different tissues, but the number of candidate enzymes which do this is small (Walchi, S., Curchod, M-L., Pescini Gobert, R., Arkinstall, S. & Hooft van Huijsduijnen, R. (2000) J. Biol. Chem. 275, 9792-9796). Thus, protein tyrosine phosphatase 1B (PTP1B) appears to be the major negative regulator of the insulin receptor in muscle and liver tissues (see for example Elechelby, M, Payette, P, Michalszyn, E, Cromlish, W, Collins, S, Loy, A L, Normandin, D, Cheng, A, Himms-Hagen, J, Chan, C-C, Ramachandran, C, Gresser, M J, Tremblay, M & Kennedy, B P (1999) Science, 283, 1544-1548; Goldstein, B J, Bittner-Kowalczyk, A, White, M F & Harbeck, M (2000) J. Biol. Chem. 275, 4283-4289). By contrast, PTP alpha may play a more dominant role in regulating the insulin receptor in adipose tissue (Calera, M R, Vallega, G & Pilch, P F (2000) J. Biol. Chem. 275 6308-6312).

The development of type 2 diabetes is characterized by a protracted period of insulin resistance. In human subjects who are obese and insulin resistant, PTP protein concentrations are increased, which has led to the idea that elevations in the proteins contributes to the cause of the diabetic state (Ahmad, F, Azevedo, J L, Cortright, R, Dohm, G L & Goldstein, B J (1997) J. Clin. Invest. 100 449-458). The two most significantly elevated are PTP1B and LAR. Considering that loss of LAR activity is associated with insulin resistance and diabetes (Ren, J-M, Li, P-M, Zhang, W-R, Sweet, L J, Cline, G, Shulman, G I, Livingston, J N & Goldstein, B J (1998) Diabetes 47 493-497), these data support the concept that PTP1B is a major contributor to the insulin resistant state and that pharmacological inhibition of its activity may go some way towards pharmaceutically alleviating the condition. Indeed, the recent reports of the knockout mouse in which PTP1B has been ablated confirm that loss of PTP1B activity leads to enhancement of the metabolic effects of insulin (Elechelby, M, Payette, P, Michalszyn, E, Cromlish, W, Collins, S, Loy, A L, Normandin, D, Cheng, A, Himms-Hagen, J, Chan, C-C, Ramachandran, C, Gresser, M J, Tremblay, M & Kennedy, B P (1999) Science, 283, 1544-1548; Klaman, L D, Ross, O, Peroni, O D, Kim, J K, Martino, J L, Zabolotny, J M, Moghal, N, Lubkin, M, Kim, Y-B, Sharpe, A H, Stricker-Krongrad, A, Shulman, G I, Neel, B G & Kahn, B B (2000) Mol. Cell. Biol. 20 5479-5489). Furthermore, inhibition of PTP 1 B with a specific small molecule has been reported to treat the symptoms of diabetes in the ob/ob mouse (Wrobel, J, Sredy, J, Moxham, C, Dietrich, A, Li, Z, Sawicki, D R, Seestaller, L, Wu. L, Katz, A, Sullivan, D, Tio, C & Zhang, Z-Y (1999) J. Med. Chem. 42 3199-3202).

WO 96/40113 discloses heterocyclic nitrogen containing compounds, such as nitropyridine or nitrothiazole, capable of inhibiting protein tyrosine phosphatase activity. Such molecules are disclosed as being useful to modulate or regulate signal transduction by inhibiting protein tyrosine phosphatase activity and to treat various disease states including diabetes mellitus.

WO 98/27065 discloses a class of compounds which are stated as being protein tyrosine phosphatase modulating compounds. These prior art compounds are however structurally distinct from the compounds claimed in the present patent application.

WO 97/08934 discloses aryl acrylic acid compounds of a certain structure, which compounds are stated as having protein tyrosine protease modulating activity. Also these prior art compounds are however structurally distinct from the compounds claimed in the present patent application.

WO 99/58519 discloses certain phenyl oxo-acetic acid compounds. These compounds are stated as being useful in the treatment of metabolic disorders related to insulin resistance and hyperglycemia. Also these prior art compounds are however structurally distinct from the compounds claimed in the present patent application.

WO 99/58521 discloses the use of 11-aryl-benzo[b]naphtho[2,3-d]furan and 11-aryl-benzo[b]naphtho[2,3-d]thiophene compounds to inhibit protein tyrosine phosphatase activity. Such compounds are disclosed as being useful to modulate or regulate signal transduction by inhibiting protein tyrosine phosphatase activity and to treat various disease states including diabetes mellitus.

The compound 6H-Pyrano[3,4-c]pyridazine-4-carbonitrile, 3-amino-2,8-dihydro-6,6-dimethyl-2-phenyl and structurally related compounds have been disclosed by E. G. Paronikyan et al. in Khim.Geterotsikl. Soedin (1996), 10, pp. 1410-1412. However, E. G. Paronikyan et al. does not disclose or even suggest that these compounds may have therapeutic activity, and particularly not in the diabetes area, such as the area of type 2 diabetes.

The object of the present invention was to provide novel compounds for the specific inhibition of PTPs allowing the study og biological processes in which they are active. Furthermore, a second object of the current invention was to provide novel compounds having improved advantages over drugs currently used for the treatment of type 2 diabetes. It should be appreciated that the wording “improved advantages” is not necessarily defined as more potent compounds, but as compounds having improved advantages overall, including but not limited to also improved selectivity and less side-effects.

### DISCLOSURE OF THE INVENTION

The novel compounds according to the present invention are defined by the general formula I

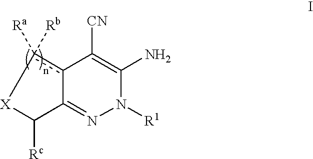

wherein

n is an integer of 1 or 2;

R¹ is

(i) phenyl or naphthyl, each optionally substituted with up to 5 substituents independently selected from the group consisting of:

(a) a straight or branched C₁-C₆ alkyl;

(b) a C₃-C₆ cycloalkyl or a C₆-C₁₀ aryl;

(c) —CO—O-(C₁-C₆ alkyl) wherein the alkyl group is straight or branched;

(d) a halogen selected from the group consisting of fluoro, chloro, bromo, and iodo;

(e) a straight or branched C₁-C₆ alkoxy;

(f) nitro;

(g) CF₃;

(h) —O-(C₁-C₆ alkyl)-phenyl wherein the alkyl group is straight or branched;

(i) a heteroaryl having 5 or 6 ring atoms, wherein 1 or 2 of the ring atoms are optionally O, N, or S, optionally substituted with one or more methyl or ethyl;

(j) a heterocycloalkyl having 5 or 6 ring atoms, wherein 1 or 2 of the ring atoms are optionally O, N, or S;

(k) —N(R²)—CO—R³, where R² is hydrogen or a straight or branched C₁-C₆ alkyl, and R³ is a straight or branched C₁-C₆ alkyl; and

(l) —N(R⁴)(R⁵), where each of R⁴ and R⁵ independently is a straight or branched C₁-C₆ alkyl;

(ii) phenyl that is fused with a cyclohexyl group or naphthyl that is fused with a cyclohexyl group, wherein 1 or 2 of the carbon atoms are optionally substituted with O, N, or S, and wherein said cyclohexyl group is optionally substituted with 1 or 2 substituents independently selected from the group consisting of halogen, CF₃, a straight or branched C₁-C₆ alkyl, a C₃-C₆ cycloalkyl, and a C₆-C₁₀ aryl; or

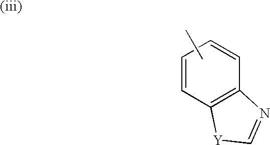

wherein Y is O, S, or N—R^{x} wherein R^{x} is a straight or branched C₁-C₆ alkyl;

X is

(i) an oxygen atom, a sulfur atom, or a methylene group;

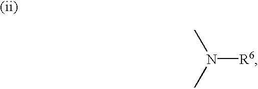

wherein R⁶ is

(a) benzyl;

(b) —CO-(C₁-C₆ alkyl) wherein the alkyl group is straight or branched;

(c) —CO—A, wherein A is phenyl, naphthyl, or a heteroaryl; said heteroaryl group having 5 to 10 ring atoms wherein at least one of said ring atoms is O, N, or S;

(d) —CO—OR^{y}, wherein R^{y} is hydrogen or a straight or branched C₁-C₆ alkyl;

(e) —CO—O-(C₁-C₆ alkyl)-A wherein A is as defined above; or

(f) —SO₂—A, wherein A is as defined above;

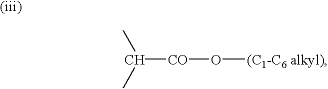

wherein the alkyl group is straight or branched; or

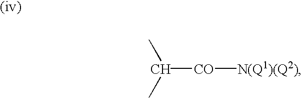

wherein each of Q¹ and Q² is independently phenyl, naphthyl, or a heteroaryl; said heteroaryl group having 5 to 10 ring atoms wherein at least one of said ring atoms is O, N, or S;

each of R^{a} and R^{b} is independently hydrogen, C₁-C₆ alkyl, or R^{a} and R^{b} together form a carbonyl group; and

R^{c} is hydrogen, a straight or branched C₁-C₆ alkyl, or carbonyl; with the proviso that the compound 6H-pyrano[3,4-c]pyridazine-4-carbonitrile, 3-amino-2,8-dihydro-6,6-dimethyl-2-phenyl is excluded.

A subset of compounds of formula I is defined by formula Ia below.

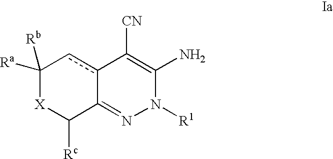

wherein X, R¹, R^{a}, R^{b} and R^{c} are as defined in formula I above.

A further subset of compounds of formula Ia is defined by formula Ia′ below.

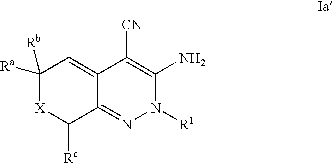

wherein X, R¹, R^{a}, R^{b} and R^{c} are as defined in formula I above.

Another subset of compounds of formula I is defined by formula Ib below.

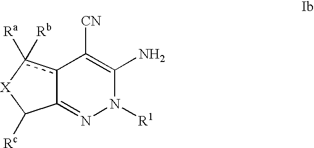

wherein X, R¹, R^{a}, R^{b} and R^{c} are as defined above.

Within the scope of the invention are also pharmaceutically and pharmacologically acceptable salts of the compounds of formula I, Ia, Ia′, and Ib, as well as hydrates thereof. Hydrochloride salts and tosylate salts of a compound of the present invention are also within the scope of the invention. Hydrochloride salts are preferred.

Some specific examples of R¹ in accordance with the present invention are phenyl or naphthyl which is optionally and independently substituted by 1, 2, 3, 4 or 5 substituents selected from the group consisting of methyl; ethyl; straight, branched or cyclic propyl, butyl, pentyl, or hexyl; —CO—O—(CH₂)ₙ—CH₃ wherein n is an integer 0, 1, 2, 3, 4, or 5; methoxy, ethoxy, propoxy, butyloxy, pentyloxy, or hexyloxy; —O—(CH₂)n-phenyl where n is an integer 0, 1, 2, 3, 4, 5, or 6.

A more specific example of R¹ is phenyl, optionally substituted with a straight or branched C₁-C₄ alkyl (e.g., methyl, ethyl, n-propyl, or isopropyl), a straight or branched C₁-C₆ alkoxy (e.g., methoxy, ethoxy, or propoxy), nitro, CF₃, halo (e.g., fluoro, chloro, or bromo; especially fluoro and chloro), cycloalkyl (e.g., cyclohexyl), heterocycloalkyl (e.g., heterocyclohexyl such as morpholino), aryl (e.g., phenyl), heteroaryl (e.g., furan), —CO—O-(C₁-C₄ alkyl) (e.g., —CO—O—CH₃ or —CO—O—CH₂CH₃), or —N(R²)—CO—R³, where R² is hydrogen, methyl, or ethyl, and R³ is a straight or branched C₁-C₄ alkyl (e.g., —NH—CO—CH₃ or —NH—CO—CH₂CH₃).

Still further specific examples of R¹ are shown below:

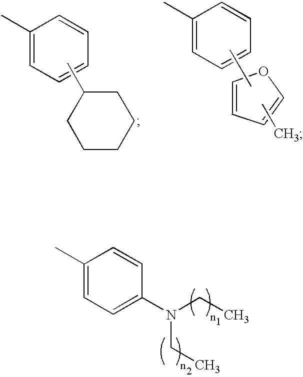

wherein each of n₁ and n₂ independently is an integer 0, 1,2,3,4, or 5;

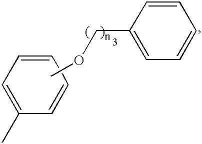

wherein n₃ is an integer 0, 1, 2, 3, 4, or 5;

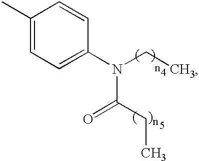

wherein each of n₄ and n₅ independently is an integer 0, 1, 2, 3, 4, or 5;

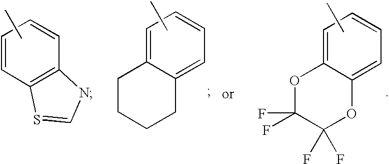

Examples of X include an oxygen atom, a sulfur atom, a methylene group,

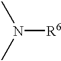

wherein R⁶ is benzyl, —CO-(C₁-C₄ alkyl), —CO—O-(C₁-C₄ alkyl), or

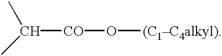

A preferred example of X is oxygen. Another preferred example of X is

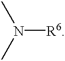

Some examples of R^{a}, R^{b}, and R^{c} are hydrogen, methyl, or ethyl.

Examples of a compound of the present invention include compounds of formula Ia′ wherein R¹ is phenyl, optionally substituted with a straight or branched C₁-C₄ alkyl, a straight or branched C₁-C₆ alkoxy, nitro, CF₃, fluoro, chloro, bromo, cyclohexyl, heterocyclohexyl, phenyl, —CO—O-(C₁-C₄ alkyl), or —N(R²)—CO—R³, where R² is hydrogen, methyl, or ethyl, and R³ is a straight or branched C₁-C₄ alkyl; X is an oxygen atom; a sulfur atom; a methylene group;

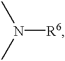

wherein R⁶ is benzyl, —CO-(C₁-C₄ alkyl), or

—CO—O-(C₁-C₄ alkyl); or

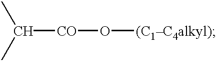

and each of R^{a}, R^{b}, and R^{c} independently is hydrogen, methyl, or ethyl.

Examples of a compound of the present invention also include compounds of formual Ia′ wherein R¹ is

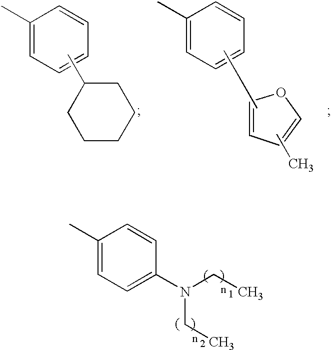

wherein each of n₁ and n₂ independently is an integer 0, 1, 2, 3, 4, or 5;

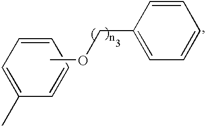

wherein n₃ is an integer 0, 1, 2, 3, 4, or 5;

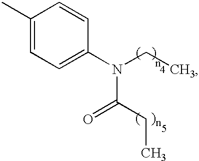

wherein each of n₄ and n₅ independently is an integer 0, 1, 2, 3, 4, or 5;

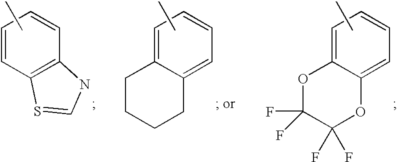

X is an oxygen atom; a sulfur atom; a methylene group;

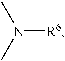

wherein R⁶ is benzyl, —CO-(C₁-C₄ alkyl), or —CO—O-(C₁-C₄ alkyl); or

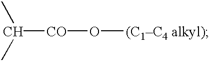

and each of R^{a}, R^{b}, and R^{c} independently is hydrogen, methyl, or ethyl.

The compounds of the present invention are useful in therapy, particular for the treatment of type 2 diabetes mellitus.

Also within the scope of the invention is the use of a compound of formula I, for the manufacture of a medicament for the treatment of type 2 diabetes mellitus.

A further aspect of the invention is the use of a compound of formula Ia (e.g., a compound of formula Ia′), for the manufacture of a medicament for the treatment of type 2 diabetes mellitus.

Still a further aspect of the invention is the use of a compound of formula Ib, for the manufacture of a medicament for the treatment of type 2 diabetes mellitus.

A further aspect of the invention is a method for the treatment of a patient suffering from type 2 diabetes mellitus, whereby an effective amount of a compound according to formula I above, is administered to a patient in need of such treatment.

A further aspect of the invention is a method for the treatment of a patient suffering from type 2 diabetes mellitus, whereby an effective amount of a compound according to formula Ia (e.g., a compound of formula Ia′) above, is administered to a patient in need of such treatment.

A further aspect of the invention is a method for the treatment of a patient suffering from type 2 diabetes mellitus, whereby an effective amount of a compound according to formula Ib above, is administered to a patient in need of such treatment.

Still a further aspect of the invention is a method for the treatment of a patient suffering from type 2 diabetes mellitus, whereby an effective amount of a compound described in each of the following Examples above, is administered to a patient in need of such treatment.

### Definitions

As used herein, an alkyl is a straight or branched hydrocarbon chain containing the indicated number of carbon atoms. Examples of alkyl groups include, but are not limited to, methyl, ethyl, propyl, isopropyl, butyl, isobutyl, sec-butyl, tert-butyl, n-pentyl, 2-methylpentyl, and n-hexyl.

By cycloalkyl is meant a cyclic alkyl group containing the indicated number of carbon atoms. Some examples of cycloalkyl are cyclopropyl, cyclopentyl, cyclohexyl, adamantyl, and norbomyl. Heterocycloalkyl is a cycloalkyl group containing the indicated number of heteroatoms such as nitrogen, oxygen, or sulfur. Examples of heterocycloalkyl include piperidinyl, piperazinyl, tetrahydropyranyl, tetrahydrofuryl, and morpholinyl.

As used herein, aryl is an aromatic group containing the indicated number of ring atoms. Examples of an aryl group include phenyl, naphthyl, phenanthryl, and anthracyl. Heteroaryl is aryl containing the indicated number of heteroatoms such as nitrogen, oxygen, or sulfur. Some examples of heteroaryl are pyridyl, furanyl, pyrrolyl, thienyl, thiazolyl, oxazolyl, and imidazolyl.

Each of the cycloalkyl, heterocycloalkyl, aryl, or heteroaryl groups described herein is optionally substituted with C₁₋₄ alkyl, C₃₋₆ cycloalkyl, 3-6 membered heterocycloalkyl, C₆₋₁₀ aryl, 6-10 membered heteroaryl, C₇₋₁₄ aralkyl, C₁₋₄ alkyl-heteroaryl with 6-10 ring atoms, C₁₋₄ alkoxy, hydroxy, hydroxyl- C₁₋₄ alkyl, carboxyl, halo, halo- C₁₋₄ alkyl, amino, amino-C₁₋₄ alkyl, nitro, cyano, C₁₋₅ alkylcarbonyloxy, C₁₋₅ alkyloxycarbonyl, C₁₋₅ alkylcarbonyl, formyl, oxo, aminocarbonyl, C₁₋₅ alkylcarbonylamino, C₁₋₄ alkylsulfonylamino, aminosulfonyl, aminocarbonyloxy, or C₁₋₄ alkyloxycarbonylamino.

Note that an amino group can be unsubstituted, mono-substituted, or di-substituted. It can be substituted with groups such as C₁₋₄ alkyl, C₃₋₆ cycloalkyl, 3-6 membered heterocycloalkyl, C₆₋₁₀ aryl, or 6-10 membered heteroaryl. Halo refers to fluoro, chloro, bromo, or iodo.

### Methods of Preparation

The compounds according to the present invention may be prepared by the following methods.

Compounds according to the present invention may be prepared by following the procedure reported by Norayan, Paronikian and Vartanyan, Khim. Geterotsikl. Soedin. pp. 1464-6 (1983) (see Scheme I and Scheme II below).

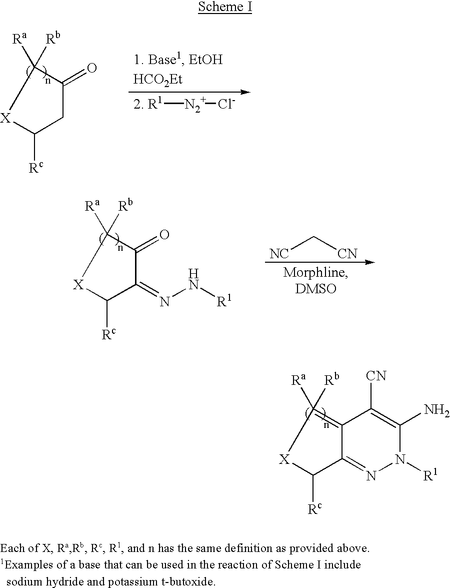

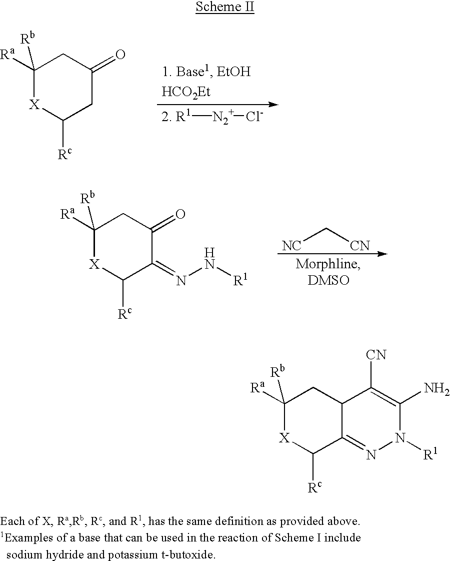

The invention will now be described in more details by the following working examples, which however should not be construed as limiting the invention.

In the following examples, NMR spectra were recorded on a Varian 400 MHz spectrometer, a Bruker Advance DPX 400 or a Bruker DRX 500 and chemical shifts are given in ppm using tetramethylsilane as an internal standard at 25° C. HPLC analyses were performed using a Waters Xterra MS C18 column (100×4.6 mm, 5μ) eluting with a gradient of 5% ACN in 95% water to 95% ACN in 5% water (0.2% TFA buffer) over 3.5 min. then 95% ACN in 5% water (0.2% TFA buffer) for a further 2.5 min. at a flow rate of 3 ml/min on a Waters 600E system with monitoring at 254 nm and on a on a Hewlett-Packard 1100 instrument with a Nucleosil C-18 column (250×4.6 mm, 3 μM) thermostated at 25° C., eluting with water (0.1% TFA)/acetonitrile at a flow rate of 1 mL/min and gradients with a 5 minute isocratic run followed by a 10 min gradient, with UV detection at 254 nm. Thin layer chromatography was carried out using pre-coated silica gel F-254 plates (thickness 0.25 mm). IR spectra were recorded on a Perkin Elmer Spectrum 1000 FTIR spectrometer. Electrospray MS spectra were obtained on a Micromass platform LCMS spectrometer. The Biotage Quad 3 system was used for parallel flash purification. Silica gel column chromatography was performed using YMC gel, silica 120 Å S-50 μm.

The following methods (i.e., Methods 1a, 1b, and 2) are employed to prepare compounds 2, 3, and 52, which are used as starting materials or intermediates in the following Examples.

Method 1a (Preparation of Compound 2)

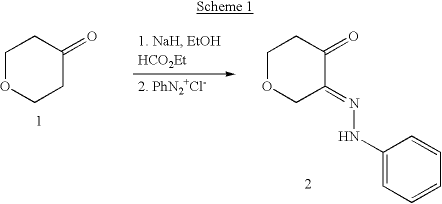

To dry ethanol (60 ml) at −20° C. was added sodium hydride (slowly) (3.92 g, 0.098 mol). Tetrahydro-4H-pyran-4-one (1) (9.79 g, 0.098 mol) was then added, followed by ethyl formate (11.9 ml, 0.147 mol) and the reaction mixture stirred at room temperature overnight. The solvent was then removed under reduced pressure and the residue dissolved in water (40 ml). The resultant solution was added to a solution of the diazonium salt of aniline, prepared by the gradual addition of a solution of sodium nitrite (6.76 g, 0.098 mol) in water (20 ml) to a solution of aniline (9.11 g, 0.098 mol) in 2 M HCl (98 ml, 0.196 mol) at −5 to 0° C. The resultant mixture was stirred at this temperature for one hour. The product (2) was then extracted with DCM, washed with water, dried (MgSO₄) and the solvent removed under reduced pressure to give a brown solid (14.5 g, 73%), the product was then recrystallized from ethanol (8.17 g, 41%); HPLC (92%, R_{T}=3.74); ¹H NMR (CDCl₃) δ 7.50-7.45 (m, Ph), 4.58 (s, CH₂), 4.01 (t, CH₂), 2.65 (t, CH₂).

Method 1b (Preparation of Compound 3)

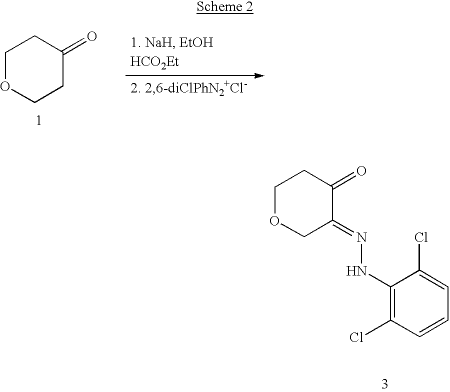

To dry ethanol (2 ml) at −20° C. was added sodium hydride (slowly) (0.1 g, 0.0025 mol). Tetrahydro-4H-pyran-4-one (0.25 g, 0.0025 mol) was then added, followed by ethyl formate (0.30 ml, 0.0038 mol) and the reaction mixture stirred at room temperature overnight. The solvent was then removed under reduced pressure and the residue dissolved in water (2 ml). The resultant solution was added to a solution of the diazonium salt of 2,6-dichloroaniline, prepared by the gradual addition of a solution of i-pentyl nitrite (0.32 g, 0.0028 mol) to a solution of 2,6-dichloroaniline (0.4 g, 0.0025 mol) in formic acid (4 ml) at 4° C. The resultant mixture was stirred at this temperature for one hour. The product was then extracted with DCM, washed with water, dried (MgSO₄) and the solvent removed under reduced pressure. This gave the hydrazone as a brown solid (0.68 g, 50%); HPLC (68%). Method 1b was carried out according to procedures described in Barbero, M. et al. Synthesis, 1171-1175 (1998).

Method 2 (Preparation of Compound 52)

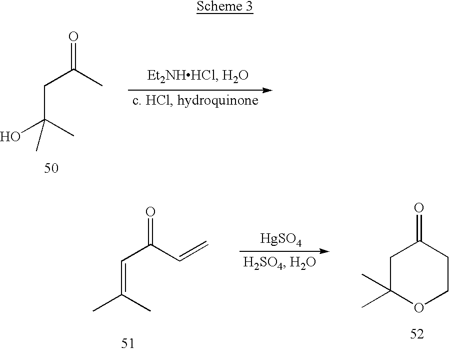

Diethylaminehydrochloride (50) (110 g, 1 mol), formaldehyde solution (78 ml, 1 mol), diacetone alcohol (125 ml, 1 mol), concentrated HCl (4 ml; for pH=1) and hydroquinone (2 g) were heated at 110° C. for three hours. The reaction mixture was then distilled under reduced pressure. The product was a green-black solution that was collected at P=14 mmHg, Tᵥₐₚ=40-100° C. The product was then re-distilled to give the divinyl ketone(51) (9.67 g, 9% yield). (P=15 mmHg, Tᵥₐₚ=70° C.; lit: b.p. 60-61° C. at 22 mmHg) NMR: δ1.95 (s, 3H); δ2.2 (s, 3H); δ5.9-6.25 (m, 4H).

Divinyl ketone (52) (11.17 g, 0.106 mol) was added dropwise to a mixture of HgSO₄ (0.86 g, 0.0029 mol), H₂SO₄ (0.86 ml) and water (34 ml) at 85° C. with stirring. Then HgSO₄ (0.251 g, 0.00073 mol) and H₂SO₄ (0.22 ml) in water (8.6 ml) were added over a period of one hour. In total the reaction mixture was heated for five hours at 85 to 100° C. This reaction mixture was then steam distilled. The product was then extracted with DCM, washed with water, dried (MgSO₄) and the solvent removed under reduced pressure to give a yellow liquid. This crude product was then purified over silica (hexane:EtOAc 2:1), which furnished 2.38 g (18%) of 2,2-dimethyl-tetrahydro-pyran-4-one (52) as a colourless oil. NMR: δ1.27 (s, 6H); δ2.36 (s, 2H); δ2.39 (t, 2H); δ4.01 (t, 2H). Method 2 was carried out according to the procedures described in J. Gen. Chem. USSR, 33(5), 1476-1480 (1963); Chem. Abs., 2161 (1949); and J. Org. Chem., 35(3), 589 (1970).

### EXAMPLES

### Example 1

Preparation of 3-amino-2-phenyl-2,8-dihydro-6H-pyrano[3,4-c]pyridazine-4-carbonitrile (compound 4)

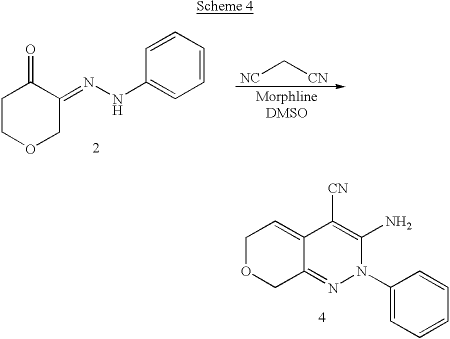

To the hydrazone (2) (1 g, 0.0049 mol) in DMSO (3 ml) was added malononitrile (0.32 g, 0.0049 mol) and morpholine (0.43 ml, 0.0049 mol) and the reaction mixture stirred at 80° C. for 15 minutes. The mixture was cooled and the product was purified by column chromatography over silica using a gradient of hexane: ethyl acetate (5:1 to 1:1). This gave the pyridazine as a brown solid (1.12 g, 91%); HPLC (94%); MS (electrospray, [M+H]⁺) m/z 253.1; ¹H NMR (DMSO) δ7.69-7.25 (m, Ph), 6.14 (s, NH₂), 5.05-5.01 (m, CH), 4.27-4.25 (m, CH₂), 4.18 (s, CH₂). The HCl salt of the pyridazine was then prepared and the compound freeze-dried to give 3-amino-2-phenyl-2,8-dihydro-6H-pyrano[3,4-c]pyridazine-4-carbonitrile; HPLC (95%, R_{T}=3.73); MS (electrospray, [M+H]⁺) m/z 253.1.

### Example 2

Preparation of 3-amino-2-phenyl-2,6,7,8-tetrahydro-4-cinnolinecarbonitrile (Compound 5)

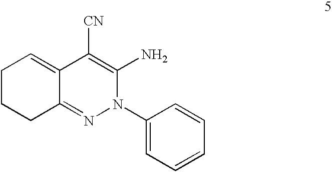

Synthetic method 1 a was used to prepare the hydrazone intermediate. The method according to Scheme 4 was undertaken to synthesize the pyridazine using the hydrazone (0.15 g, 0.0007 mol), DMSO (3 ml), malononitrile (0.05 g, 0.0007 mol) and morpholine (0.064 ml, 0.0007 mol). The reaction mixture was stirred at 80° C. for 15 minutes. The mixture was cooled and the solution poured into cold water (20 ml). The resultant precipitate was collected by filtration, recrystallized from ethanol, then freeze-dried. This gave 3-amino-2-phenyl-2,6,7,8-tetrahydro-4-cinnolinecarbonitrile as a brown solid (0.107 g, 58%); HPLC (91%, R_{T}=4.04); MS (electrospray, [M+H]⁺) m/z 251.2; ¹H NMR (CDCl₃) δ7.49-7.24 (m, Ph), 5.28 (t, CH), 4.43 (s, NH₂), 2.38-2.42 (m, CH₂), 2.22-2.25 (m, CH₂), 1.77-1.80 (m, CH₂).

### Example 3

Preparation of 3-amino-2-phenyl-6,7-dihydro-2H-cyclopenta[c]pyridazine-4-carbonitrile (Compound 6)

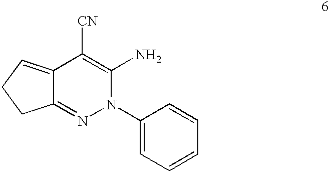

Synthetic method 1a was used to prepare the hydrazone intermediate. The method according to Scheme 4 was undertaken to synthesize the pyridazine using the hydrazone (0.5 g, 0.0027 mol), DMSO (1 ml), malononitrile (0.18 g, 0.0027 mol) and morpholine (0.23 ml, 0.0027 mol). The reaction mixture was stirred at 80° C. for 15 minutes. The mixture was cooled and the product was purified by column chromatography over silica using a gradient of hexane : ethyl acetate (5:1 to 1:1), then freeze-dried. This gave 3-amino-2-phenyl-6,7-dihydro-2H-cyclopenta[c]pyridazine-4-carbonitrile as a green solid (0.33 g, 53%); HPLC (93%, R_{T}=3.79); MS (electrospray, [M+H]⁺) m/z 237.0.

### Example 4

Preparation of 3-amino-7-benzyl-2-phenyl-2,6,7,8-tetrahydropyrido[3,4-c]pyridazine-4-carbonitrile (Compound 7)

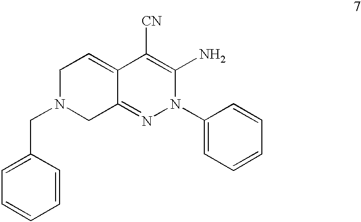

Synthetic method 1 a was used to prepare the hydrazone intermediate. The method according to Scheme 4 was undertaken to synthesize the pyridazine using the hydrazone (0.2 g, 0.0007 mol), DMSO (1 ml), malononitrile (0.05 g, 0.0007 mol) and morpholine (0.06 ml, 0.0007 mol). The reaction mixture was stirred at 80° C. for 15 minutes. The mixture was cooled and the product was purified by column chromatography over silica using a gradient of hexane : ethyl acetate (4:1 to ethyl acetate), then freeze-dried. This gave 3-amino-7-benzyl-2-phenyl-2,6,7,8-tetrahydropyrido[3,4-c]pyridazine-4-carbonitrile as an orange solid (0.07 g, 30%); HPLC (82%, R_{T}=3.88); MS (electrospray, [M+H]⁺) m/z 342.0.

### Example 5

Preparation of 7-acetyl-3-amino-2-phenyl-2,6,7,8-tetrahydropyrido[3,4-c]pyridazine-4-carbonitrile (Compound 8)

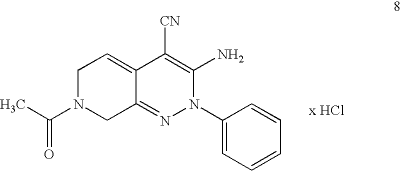

Synthetic method 1a was used to prepare the hydrazone intermediate. The method described in Scheme 4 was undertaken to synthesize the pyridazine using the hydrazone (0.5 g, 0.0021 mol), DMSO (1 ml), malononitrile (0.14 g, 0.0021 mol) and morpholine (0.18 ml, 0.0021 mol). The reaction mixture was stirred at 80° C. for 15 minutes. The mixture was cooled and the product was purified by column chromatography over silica using a gradient of hexane : ethyl acetate (4:1 to ethyl acetate), then freeze-dried. This gave 7-acetyl-3-amino-2-phenyl-2,6,7,8-tetrahydropyrido[3,4-c]pyridazine-4-carbonitrile as an orange solid (0.09 g, 15%); HPLC (95%, R_{T}=3.49); MS (electrospray, [M+H]⁺) m/z 294.1.

### Example 6

Preparation of 3-amino-2-(2-fluorophenyl)-2,8-dihydro-6H-pyrano [3,4-c]pyridazine-4-carbonitrile hydrochloride (Compound 9)

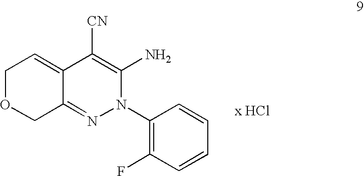

To the hydrazone (0.405 g, 0.0018 mol) in DMSO (1 ml) was added malononitrile (0.12 g, 0.0018 mol) and morpholine (0.16 ml, 0.0018 mol) and the reaction mixture stirred at 80° C. for 15 minutes. The mixture was cooled and the product was purified by column chromatography over silica using hexane: ethyl acetate (1:1). This gave the pyridazine as a green solid (0.156 g, 32%); HPLC (97%); MS (electrospray, [M+H]⁺) m/z 271.1. The HCl salt of the pyridazine was then prepared and the compound freeze-dried to give 3-amino-2-(2-fluorophenyl)-2,8-dihydro-6H-pyrano[3,4-c]pyridazine-4-carbonitrile hydrochloride; HPLC (97%, R_{T}=3.81); MS (electrospray, [M+H]⁺) m/z 271.1.

### Example 7

Preparation of 3-amino-2-(4-methylphenyl)-2,8-dihydro-6H-pyrano[3,4-c]pyridazine-4-carbonitrile hydrochloride (Compound 10)

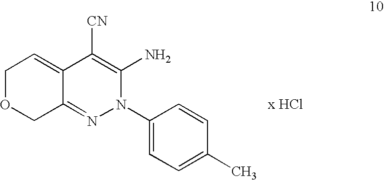

To the hydrazone (0.392 g, 0.0018 mol) in DMSO (1 ml) was added malononitrile (0.12 g, 0.0018 mol) and morpholine (0.16 ml, 0.0018 mol) and the reaction mixture stirred at 80° C. for 15 minutes. The mixture was cooled and the product was purified by column chromatography over silica using hexane : ethyl acetate (2:1). This gave the pyridazine as a green solid (0.346 g, 72%); HPLC (96%); MS (electrospray, [M+H]⁺) m/z 267.1. The HCl salt of the pyridazine was then prepared and the compound freeze-dried to give 3-amino-2-(4-methylphenyl)-2,8-dihydro-6H-pyrano[3,4-c]pyridazine-4-carbonitrile hydrochloride; HPLC (96%, R_{T}=3.96); MS (electrospray, [M+H]⁺) m/z 267.1.

### Example 8

Preparation of 3-amino-2-[3-(trifluoromethyl)phenyl]-2,8-dihydro-6H-pyrano [3,4-c]pyridazine-4-carbonitrile Hydrochloride (Compound 11)

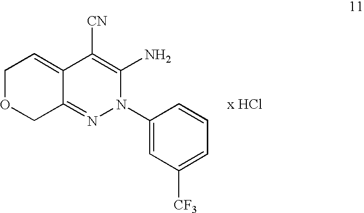

To the hydrazone (0.622 g, 0.0023 mol) in DMSO (1 ml) was added malononitrile (0.15 g, 0.0023 mol) and morpholine (0.2 ml, 0.0023 mol) and the reaction mixture stirred at 80° C. for 15 minutes. The mixture was cooled and the product was purified by column chromatography over silica using hexane : ethyl acetate (2:1). This gave the pyridazine as a green/brown solid; HPLC (100%); MS (electrospray, [M+H]⁺) m/z 321.1. The HCl salt of the pyridazine was then prepared and the compound freeze-dried to give 3-amino-2-[3-(trifluoromethyl)phenyl]-2,8-dihydro-6H-pyrano[3,4-c]pyridazine-4-carbonitrile hydrochloride (0.747 g, 91%); HPLC (100%, R_{T}=4.26); MS (electrospray, [M+H]⁺) m/z 321.1.

### Example 9

Preparation of 3-amino-2-phenyl-2,8-dihydro-6H-thiopyrano[3,4-c]pyridazine-4-carbonitrile Hydrochloride (Compound 12)

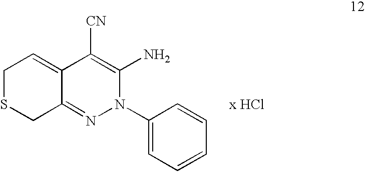

To the hydrazone (0.1 g, 0.00045 mol) in DMSO (0.3 ml) was added malononitrile (0.03 g, 0.00045 mol) and morpholine (0.04 ml, 0.00045 mol) and the reaction mixture stirred at 80° C. for 15 minutes. The mixture was cooled and the product was purified by column chromatography over silica using a gradient of hexane : ethyl acetate (4:1 to 1:1). This gave the pyridazine as a green solid; HPLC (82%); MS (electrospray, [M+H]⁺) m/z 269.0. The HCl salt of the pyridazine was then prepared and the compound freeze-dried to give 3-amino-2-phenyl-2,8-dihydro-6H-thiopyrano[3,4-c]pyridazine-4-carbonitrile hydrochloride (0.086 g, 63%); HPLC (82%, R_{T}=3.75); MS (electrospray, [M+H]⁺) m/z 269.0.

### Example 10

Preparation of 3-amino-2-(3-methoxyphenyl)-2,8-dihydro-6H-pyrano[3,4-c]pyridazine-4-carbonitrile Hydrochloride (Compound 13)

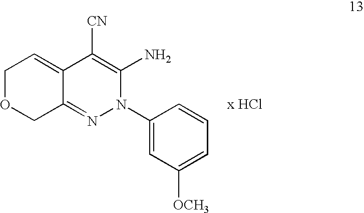

To the hydrazone (0.474 g, 0.002 mol) in DMSO (2 ml) was added malononitrile (0.132 g, 0.002 mol) and morpholine (0.18 ml, 0.002 mol) and the reaction mixture stirred at 80° C. for 15 minutes. The mixture was cooled and the product was purified by column chromatography over silica using a gradient of hexane : ethyl acetate (2:1 to 1:1). This gave the pyridazine as a green solid (0.15 g, 27%); HPLC (93%); MS (electrospray, [M+H]⁺) m/z 283.1. The HCl salt of the pyridazine was then prepared and the compound freeze-dried to give 3-amino-2-(3-methoxyphenyl)-2,8-dihydro-6H-pyrano[3,4-c]pyridazine-4-carbonitrile hydrochloride; HPLC (93%, R_{T}=2.37); MS (electrospray, [M+H]⁺) m/z 283.1.

### Example 11

Preparation of 3-amino-2-(2,6-dimethylphenyl)-2,8-dihydro-6H-pyrano[3,4-c]pyridazine-4-carbonitrile Hydrochloride (Compound 14)

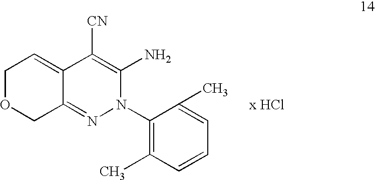

To the hydrazone (0.45 g, 0.0019 mol) in DMSO (2 ml) was added malononitrile (0.13 g, 0.0019 mol) and morpholine (0.17 ml, 0.0019 mol) and the reaction mixture stirred at 80° C. for 15 minutes. The mixture was cooled and the product was purified by column chromatography over silica using hexane: ethyl acetate (2:1). This gave the pyridazine as a green solid; HPLC (97%); MS (electrospray, [M+H]⁺) m/z 281.1. The HCl salt of the pyridazine was then prepared and the compound freeze-dried to give 3-amino-2-(2,6-dimethylphenyl)-2,8-dihydro-6H-pyrano[3,4-c]pyridazine-4-carbonitrile hydrochloride (0.563 g, 94%); HPLC (97%, R_{T}=3.42); MS (electrospray, [M+H]⁺) m/z 281.1.

### Example 12

Preparation of Ethyl 3-amino-4-cyano-2-phenyl-2,6,7,8-tetrahydro-7-cinnolinecarboxylate Hydrochloride (Compound 15)

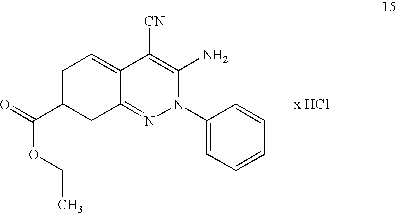

To the hydrazone (0.2 g, 0.00073 mol) in DMSO (0.6 ml) was added malononitrile (0.048 g, 0.00073 mol) and morpholine (0.064 ml, 0.00073 mol) and the reaction mixture stirred at 80° C. for 15 minutes. The mixture was cooled and the product was purified by column chromatography over silica using a gradient of hexane : ethyl acetate (5:1 to 1:1). This gave the pyridazine as a brown solid; HPLC (92%); MS (electrospray, [M+H]⁺) m/z 323.1. The HCl salt of the pyridazine was then prepared and the compound freeze-dried to give ethyl 3-amino-4-cyano-2-phenyl-2,6,7,8-tetrahydro-7-cinnolinecarboxylate hydrochloride (0.052 g, 22%); HPLC (92%, R_{T}=3.86); MS (electrospray, [M+H]⁺) m/z 323.1.

### Example 13

Preparation of 3-amino-2-(3,4-dichlorophenyl)-2,8-dihydro-6H-pyrano[3,4-c]pyridazine-4-carbonitrile Hydrochloride (Compound 16)

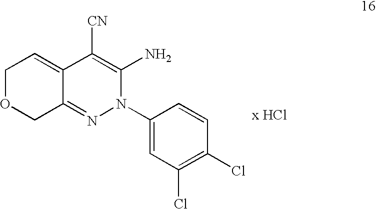

To the hydrazone (0.3 g, 0.0011 mol) in DMSO (2 ml) was added malononitrile (0.073 g, 0.0011 mol) and morpholine (0.1 ml, 0.0011 mol) and the reaction mixture stirred at 80° C. for 15 minutes. The mixture was cooled and the product was purified by column chromatography over silica using a gradient of hexane: ethyl acetate (4:1 to 2:1). This gave the pyridazine as a green solid; HPLC (94%); MS (electrospray, [M+H]⁺) m/z 321.1. The HCl salt of the pyridazine was then prepared and the compound freeze-dried to give 3-amino-2-(3,4-dichlorophenyl)-2,8-dihydro-6H-pyrano[3,4-c]pyridazine-4-carbonitrile hydrochloride (0.127 g, 32%); HPLC (94%, R_{T}=3.47); MS (electrospray, [M+H]⁺) m/z 321.1.

### Example 14

Preparation of 7-benzoyl-3-amino-2-phenyl-2,6,7,8-tetrahydropyrido[3,4-c]pyridazine-4-carbonitrile (Compound 17)

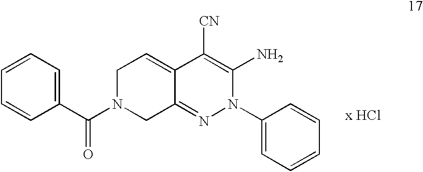

To the hydrazone (0.37 g, 0.00121 mol) in DMSO (2 ml) was added malononitrile (0.08 g, 0.00121 mol) and morpholine (0.11 ml, 0.00121 mol) and the reaction mixture stirred at 80° C. for 15 minutes. The mixture was cooled and the product was purified by column chromatography over silica using a gradient of hexane : ethyl acetate (1:1 to ethyl acetate). This gave the pyridazine as a green solid (0.11 g, 26%); HPLC (91%); MS (electrospray, [M+H]⁺) m/z 356.4. The HCl salt of the pyridazine was then prepared and the compound freeze-dried to give 7-benzoyl-3-amino-2-phenyl-2,6,7,8-tetrahydropyrido[3,4-c]pyridazine-4-carbonitrile; HPLC (91%, R_{T}=4.45); MS (electrospray, [M+H]⁺) m/z 356.4.

### Example 15

Preparation of 3-amino-2-(4-methoxyphenyl)-2,8-dihydro-6H-pyrano[3,4-c]pyridazine-4-carbonitrile Hydrochloride (Compound 18)

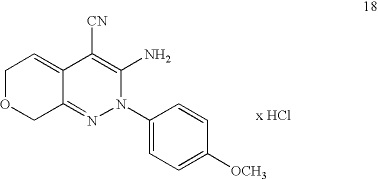

To the hydrazone (0.131 g, 0.00055 mol) in DMSO (1 ml) was added malononitrile (0.037 g, 0.00055 mol) and morpholine (0.049 ml, 0.00055 mol) and the reaction mixture stirred at 80° C. for 15 minutes. The mixture was cooled and the product was purified by column chromatography over silica using a gradient of hexane : ethyl acetate (4:1 to ethyl acetate). This gave the pyridazine as a green solid (0.095 g, 61%); HPLC (98%); MS (electrospray, [M+H]⁺) m/z 283.3. The HCl salt of the pyridazine was then prepared and the compound freeze-dried to give 3-amino-2-(4-methoxyphenyl)-2,8-dihydro-6H-pyrano[3,4-c]pyridazine-4-carbonitrile hydrochloride; HPLC (98%, R_{T}=4.21); MS (electrospray, [M+H]⁺) m/z 283.3.

### Example 16

Preparation of 3-amino-2-(4-isopropylphenyl)-2,8-dihydro-6H-pyrano[3,4-c]pyridazine-4-carbonitrile Hydrochloride (Compound 19)

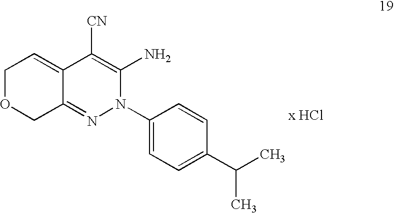

To the hydrazone (0.25 g, 0.001 mol) in DMSO (1 ml) was added malononitrile (0.067 g, 0.001 mol) and morpholine (0.089 ml, 0.001 mol) and the reaction mixture stirred at 80° C. for 15 minutes. The mixture was cooled and the product was purified by column chromatography over silica using hexane : ethyl acetate (3:1). This gave the pyridazine as a green solid (0.103 g, 35%); HPLC (94%); MS (electrospray, [M+H]⁺) m/z 295.3. The HCl salt of the pyridazine was then prepared and the compound freeze-dried to give 3-amino-2-(4-isopropylphenyl)-2,8-dihydro-6H-pyrano[3,4-c]pyridazine-4-carbonitrile hydrochloride; HPLC (94%, R_{T}=4.72); MS (electrospray, [M+H]⁺) m/z 295.3.

### Example 17

Preparation of 3-amino-2- [4-(4-morpholinyl)phenyl] -2,8-dihydro-6H-pyrano [3,4-c]pyridazine-4-carbonitrile hydrochloride (Compound 20)

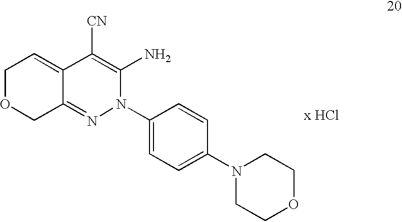

To the hydrazone (0.1 g, 0.00035 mol) in DMSO (1 ml) was added malononitrile (0.023 g, 0.00035 mol) and morpholine (0.03 ml, 0.00035 mol) and the reaction mixture stirred at 80° C. for 15 minutes. The mixture was cooled and the product was purified by column chromatography over silica using a gradient of hexane : ethyl acetate (2:1 to ethyl acetate). This gave the pyridazine as a brown solid (0.026 g, 19%); HPLC (95%); MS (electrospray, [M+H]⁺) m/z 338.5. The HCl salt of the pyridazine was then prepared and the compound freeze-dried to give 3-amino-2-[4-(4-morpholinyl)phenyl]-2,8-dihydro-6H-pyrano[3,4-c]pyridazine-4-carbonitrile hydrochloride; HPLC (95%, R_{T}=4.32); MS (electrospray, [M+H]⁺) m/z 338.5.

### Example 18

Preparation of 7-acetyl-3-amino-2-(2,6-dimethylphenyl)-2,6,7,8-tetrahydropyrido[3,4-c]pyridazine-4-carbonitrile Hydrochloride (Compound 21)

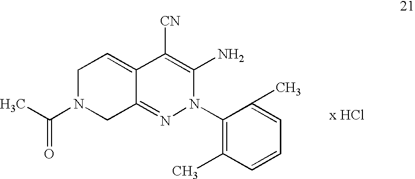

To the hydrazone (0.33 g, 0.0012 mol) in DMSO (2 ml) was added malononitrile (0.08 g, 0.0012 mol) and morpholine (0.11 ml, 0.0012 mol) and the reaction mixture stirred at 80° C. for 15 minutes. The mixture was cooled and the product was purified by column chromatography over silica using a gradient of hexane : ethyl acetate (1:1 to ethyl acetate). This gave the pyridazine as a brown solid (0.11 g, 28%); HPLC (84%); MS (electrospray, [M+H]⁺) m/z 322.4. The HCl salt of the pyridazine was then prepared and the compound freeze-dried to give 7-acetyl-3-amino-2-(2,6-dimethylphenyl)-2,6,7,8-tetrahydropyrido[3,4-c]pyridazine-4-carbonitrile hydrochloride; HPLC (84%, R_{T}=4.58); MS (electrospray, [M+H]⁺) m/z 322.4.

### Example 19

Preparation of ethyl 3-(3-amino-4-cyano-6H-pyrano[3,4-c]pyridazin-2(8H)-yl)benzoate Hydrochloride (Compound 22)

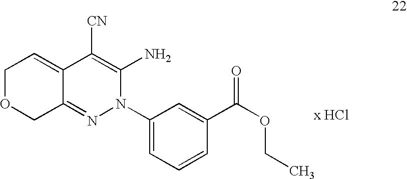

To the hydrazone (0.125 g, 0.00045 mol) in DMSO (0.5 ml) was added malononitrile (0.03 g, 0.00045 mol) and morpholine (0.04 ml, 0.00045 mol) and the reaction mixture stirred at 80° C. for 15 minutes. The mixture was cooled and the product was purified by column chromatography over silica using a gradient of hexane: ethyl acetate (4:1 to 1:1). This gave the pyridazine as a green solid (0.133 g, 91%); HPLC (97%); MS (electrospray, [M+H]⁺) m/z 325.3. The HCl salt of the pyridazine was then prepared and the compound freeze-dried to give ethyl 3-(3-amino-4-cyano-6H-pyrano[3,4-c] pyridazin-2(8H)-yl)benzoate hydrochloride; HPLC (97%, R_{T}=4.68); MS (electrospray, [M+H]⁺) m/z 325.3.

### Example 20

Preparation of Ethyl 3-amino-4-cyano-2-phenyl-2,8-dihydropyrido[3,4-c]pyridazine-7(6H)-carboxylate Hydrochloride (Compound 23)

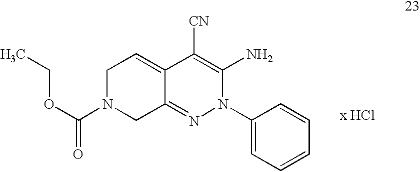

To the hydrazone (0.305 g, 0.0011 mol) in DMSO (2 ml) was added malononitrile (0.073 g, 0.0011 mol) and morpholine (0.09 ml, 0.0011 mol) and the reaction mixture stirred at 80° C. for 15 minutes. The mixture was cooled and the product was purified by column chromatography over silica using a gradient of hexane : ethyl acetate (4:1 to 1:1). This gave the pyridazine as a green solid (0.14 g, 40%); HPLC (94%); MS (electrospray, [M+H]⁺) m/z 324.4. The HCl salt of the pyridazine was then prepared and the compound freeze-dried to give ethyl 3-amino-4-cyano-2-phenyl-2,8-dihydropyrido[3,4-c]pyridazine-7(6H)-carboxylate hydrochloride; HPLC (94%, R_{T}=4.52); MS (electrospray, [M+H]⁺) m/z 324.4.

### Example 21

Preparation of 3-amino-2-(2-chloro-6-fluorophenyl)-2,8-dihydro-6H-pyrano[3,4-c]pyridazine-4-carbonitrile Hydrochloride (Compound 24)

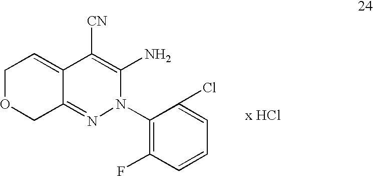

To the hydrazone (0.46 g, 0.0018 mol) in DMSO (0.5 ml) was added malononitrile (0.12 g, 0.0018 mol) and morpholine (0.16 ml, 0.0018 mol) and the reaction mixture stirred at 80° C. for 15 minutes. The mixture was cooled and the product was purified by column chromatography over silica using a gradient of hexane: ethyl acetate (4:1 to 1:1). This gave the pyridazine as a green solid (0.224 g, 41%); HPLC (98%); MS (electrospray, [M+H]⁺) m/z 305.2. The HCl salt of the pyridazine was then prepared and the compound freeze-dried to give 3-amino-2-(2-chloro-6-fluorophenyl)-2,8-dihydro-6H-pyrano[3,4-c]pyridazine-4-carbonitrile hydrochloride; HPLC (98%, R_{T}=4.45); MS (electrospray, [M+H]⁺) m/z 305.2.

### Example 22

Preparation of 3-amino-2-(2-bromo-5-trifluoromethylphenyl)-2,8-dihydro-6H-pyrano[3,4-c]pyridazine-4-carbonitrile Hydrochloride (Compound 25)

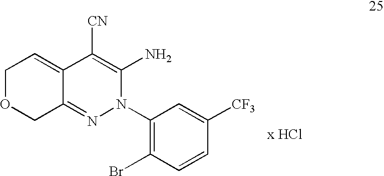

To the hydrazone (0.1 g, 0.00027 mol) in DMSO (0.5 ml) was added malononitrile (0.02 g, 0.00027 mol) and morpholine (0.024 ml, 0.00027 mol) and the reaction mixture stirred at 80° C. for 15 minutes. The mixture was cooled and the product was purified by column chromatography over silica using a gradient of hexane : ethyl acetate (2:1 to 1:1). This gave the pyridazine as a green solid; HPLC (95%); MS (electrospray, [M+H]⁺) m/z 401.2. The HCl salt of the pyridazine was then prepared and the compound freeze-dried to give 3-amino-2-(2-bromo-5-trifluoromethylphenyl)-2,8-dihydro-6H-pyrano[3,4-c]pyridazine-4-carbonitrile hydrochloride (0.084 g, 70%); HPLC (95%, R_{T}=4.31); MS (electrospray, [M+H]⁺) m/z 401.2.

### Example 23

Preparation of 3-amino-2-(1,3-benzothiazol-6-yl)-2,8-dihydro-6H-pyrano[3,4-c]pyridazine-4-carbonitrile Hydrochloride (Compound 26)

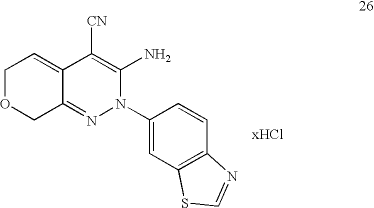

To the hydrazone (0.072 g, 0.00028 mol) in DMSO (0.5 ml) was added malononitrile (0.018 g, 0.00028 mol) and morpholine (0.024 ml, 0.00028 mol) and the reaction mixture stirred at 80° C. for 15 minutes. The mixture was cooled and the product was purified by column chromatography over silica using a gradient of hexane : ethyl acetate (2:1 to ethyl acetate). This gave the pyridazine as a beige solid; HPLC (91%); MS (electrospray, [M+H]⁺) m/z 310.3. The HCl salt of the pyridazine was then prepared and the compound freeze-dried to give 3-amino-2-(1,3-benzothiazol-6-yl)-2,8-dihydro-6H-pyrano[3,4-c]pyridazine-4-carbonitrile hydrochloride (0.06 g, 63%); HPLC (91%, R_{T}=3.66); MS (electrospray, [M+H]⁺) m/z 310.3.

### Example 24

Preparation of N-[4-(3-amino-4-cyano-6H-pyrano[3,4-c]pyridazin-2(8H)-yl)phenyl]-N-methylacetamide Hydrochloride (Compound 27)

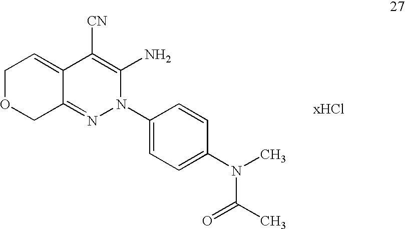

To the hydrazone (0.37 g, 0.0014 mol) in DMSO (0.5 ml) was added malononitrile (0.089 g, 0.0014 mol) and morpholine (0.12 ml, 0.0014 mol) and the reaction mixture stirred at 80° C. for 15 minutes. The mixture was cooled and the product was purified by column chromatography over silica using a gradient of hexane : ethyl acetate (2:1 to ethyl acetate). This gave the pyridazine as a brown solid; HPLC (100%); MS (electrospray, [M+H]⁺) m/z 324.4. The HCl salt of the pyridazine was then prepared and the compound freeze-dried to give N-[4-(3-amino-4-cyano-6H-pyrano[3,4-c]pyridazin-2(8H)-yl)phenyl]-N-methylacetamide hydrochloride (0.097 g, 20%); HPLC (100%, R_{T}=3.58); MS (electrospray, [M+H]⁺) m/z 324.4.

### Example 25

Preparation of 3-amino-2-[4-(dimethylamino)phenyl]-2,8-dihydro-6H-pyrano[3,4-c]pyridazine-4-carbonitrile Hydrochloride (Compound 28)

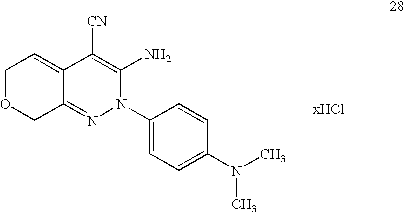

To the hydrazone (0.26 g, 0.0011 mol) in DMSO (0.5 ml) was added malononitrile (0.07 g, 0.0011 mol) and morpholine (0.09 ml, 0.0011 mol) and the reaction mixture stirred at 80° C. for 15 minutes. The mixture was cooled and the product was purified by column chromatography over silica using a gradient of hexane : ethyl acetate (2:1 to 1:1). This gave the pyridazine as a green solid; HPLC (91%); MS (electrospray, [M+H]⁺) m/z 296.4. The HCl salt of the pyridazine was then prepared and the compound freeze-dried to give 3-amino-2-[4-(dimethylamino)phenyl]-2,8-dihydro-6H-pyrano[3,4-c]pyridazine-4-carbonitrile hydrochloride (0.039 g, 11%); HPLC (91%, R_{T}=3.87); MS (electrospray, [M+H]⁺) m/z 296.4.

### Example 26

Preparation of 3-amino-2-(4-chloro-2-methylphenyl)-2,8-dihydro-6H-pyrano[3,4-c]pyridazine-4-carbonitrile Hydrochloride (Compound 29)

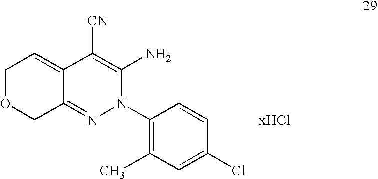

To the hydrazone (0.061 g, 0.00024 mol) in DMSO (0.5 ml) was added malononitrile (0.016 g, 0.00024 mol) and morpholine (0.021 ml, 0.00024 mol) and the reaction mixture stirred at 80° C. for 15 minutes. The mixture was cooled and the product was purified by column chromatography over silica using a gradient of hexane : ethyl acetate (2:1 to 1:1), then repeated over alumina. This gave 3-amino-2-(4-chloro-2-methylphenyl)-2,8-dihydro-6H-pyrano[3,4-c]pyridazine-4-carbonitrile hydrochloride as a green solid (0.016 g, 22%); HPLC (91%, R_{T}=4.08); MS (electrospray, [M+H]⁺) m/z 301.3.

### Example 27

Preparation of 3-amino-2-(2-chloro-6-methylphenyl)-2,8-dihydro-6H-pyrano[3,4-c]pyridazine-4-carbonitrile Hydrochloride (Compound 30)

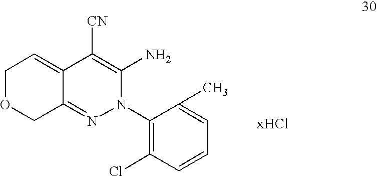

To the hydrazone (0.87 g, 0.0034 mol) in DMSO (0.5 ml) was added malononitrile (0.23 g, 0.0034 mol) and morpholine (0.3 ml, 0.0034 mol) and the reaction mixture stirred at 80° C. for 15 minutes. The mixture was cooled and the product was purified by column chromatography over silica using hexane:ethyl acetate (1:1), then repeated over alumina. This gave the pyridazine as a green solid (0.5 g, 49%); HPLC (96%); MS (electrospray, [M+H]⁺) m/z 301.3. The HCl salt of the pyridazine was then prepared and the compound freeze-dried to give 3-amino-2-(2-chloro-6-methylphenyl)-2,8-dihydro-6H-pyrano[3,4-c]pyridazine-4-carbonitrile hydrochloride; HPLC (96%, 3.12); MS (electrospray, [M+H]⁺) m/z 301.3.

### Example 28

Preparation of 3-amino-2-(2-methoxyphenyl)-2,8-dihydro-6H-pyrano[3,4-c]pyridazine-4-carbonitrile Hydrochloride (Compound 31)

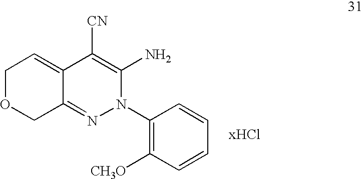

To the hydrazone (0.546 g, 0.0023 mol) in DMSO (0.5 ml) was added malononitrile (0.154 g, 0.0023 mol) and morpholine (0.2 ml, 0.0023 mol) and the reaction mixture stirred at 80° C. for 15 minutes. The mixture was cooled and the product was purified by column chromatography over silica using a gradient of hexane:ethyl acetate (2:1 to ethyl acetate). This gave the pyridazine as a pale green solid (0.265 g, 41%); HPLC (99%); MS (electrospray, [M+H]⁺) m/z 283.3. The HCl salt of the pyridazine was then prepared and the compound freeze-dried to give 3-amino-2-(2-methoxyphenyl)-2,8-dihydro-6H-pyrano[3,4-c]pyridazine-4-carbonitrile hydrochloride (0.084 g, 70%); HPLC (99%, R_{T}=4.35); MS (electrospray, [M+H]⁺) m/z 283.3.

### Example 29

Preparation of 3-amino-2-mesityl-2,8-dihydro-6H-pyrano[3,4-c]pyridazine-4-carbonitrile Hydrochloride (Compound 32)

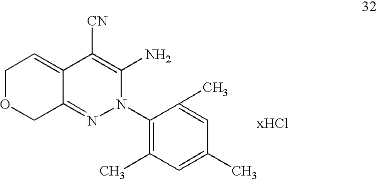

To the hydrazone (1.82 g, 0.0074 mol) in DMSO (0.5 ml) was added malononitrile (0.49 g, 0.0074 mol) and morpholine (0.65 ml, 0.0074 mol) and the reaction mixture stirred at 80° C. for 15 minutes. The mixture was cooled and the product was purified by column chromatography over silica using a gradient of hexane:ethyl acetate (1:1 to ethyl acetate). This gave the pyridazine as a green solid (0.262 g, 12%); HPLC (91%); MS (electrospray, [M+H]⁺) m/z 295.3. The HCl salt of the pyridazine was then prepared and the compound freeze-dried to give 3-amino-2-mesityl-2,8-dihydro-6H-pyrano[3,4-c]pyridazine-4-carbonitrile hydrochloride; HPLC (91%, R_{T}=5.73); MS (electrospray, [M+H]⁺) m/z 295.3.

### Example 30

Preparation of 3-amino-2-(2-isopropylphenyl)-2,8-dihydro-6H-pyrano[3,4-c]pyridazine-4-carbonitrile Hydrochloride (Compound 33)

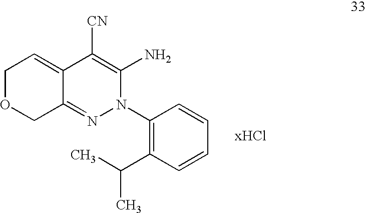

To the hydrazone (4.6 g, 0.0187 mol) in DMSO (1 ml) was added malononitrile (1.24 g, 0.0187 mol) and morpholine (1.63 ml, 0.0187 mol) and the reaction mixture stirred at 80° C. for 15 minutes. The mixture was cooled and the product was purified by column chromatography over silica using a gradient of hexane:ethyl acetate (1:1 to ethyl acetate). This gave the pyridazine as a brown solid; HPLC (93%); MS (electrospray, [M+H]⁺) m/z 295.3. The HCl salt of the pyridazine was then prepared and the compound freeze-dried to give 3-amino-2-(2-isopropylphenyl)-2,8-dihydro-6H-pyrano[3,4-c]pyridazine-4-carbonitrile hydrochloride (1.5 g, 27%); HPLC (91%, R_{T}=3.13); MS (electrospray, [M+H]⁺) m/z 295.3.

### Example 31

Preparation of 3-amino-2-(2,6-dichlorophenyl)-2,8-dihydro-6H-pyrano[3,4-c]pyridazine-4-carbonitrile Hydrochloride (Compound 34)

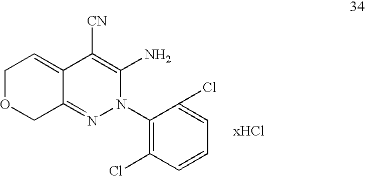

To the hydrazone (0.68 g, 0.0025 mol) in DMSO (0.5 ml) was added malononitrile (0.16 g, 0.0025 mol) and morpholine (0.22 ml, 0.0025 mol) and the reaction mixture stirred at 80° C. for 15 minutes. The mixture was cooled and the product was purified by column chromatography over silica using a gradient of hexane:ethyl acetate (2:1 to 1:1), then repeated over alumina. This gave the pyridazine as a green solid (0.074 g, 9%); HPLC (93%); MS (electrospray, [M+H]⁺) m/z 321.3. The HCl salt of the pyridazine was then prepared and the compound freeze-dried to give 3-amino-2-(2,6-dichlorophenyl)-2,8-dihydro-6H-pyrano[3,4-c]pyridazine-4-carbonitrile hydrochloride; HPLC (93%, R_{T}=2.96); MS (electrospray, [M+H]⁺) m/z 321.3.

### Example 32

Preparation of 3-amino-2-(2-methyl-4-nitrophenyl)-2,8-dihydro-6H-pyrano[3,4-c]pyridazine-4-carbonitrile tosylate (compound 35)

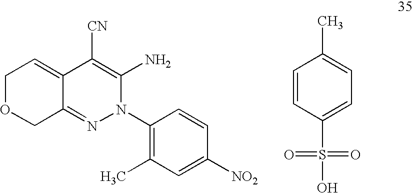

To the hydrazone (0.9 g, 0.0034 mol) in DMSO (0.5 ml) was added malononitrile (0.23 g, 0.0034 mol) and morpholine (0.3 ml, 0.0034 mol) and the reaction mixture stirred at room temperature for 15 minutes. The product was purified by column chromatography over silica using a gradient of hexane: ethyl acetate (2:1 to 1:1), then repeated over alumina. This gave the pyridazine as a green solid (0.105 g, 10%); HPLC (87%); MS (electrospray, [M+H]⁺) m/z 312.4. The tosylate salt of the pyridazine was then prepared and the compound freeze-dried to give 3-amino-2-(2-methyl-4-nitrophenyl)-2,8-dihydro-6H-pyrano[3,4-c]pyridazine-4-carbonitrile tosylate; HPLC (77%, R_{T}=2.96); MS (electrospray, [M+H]⁺) m/z 312.4.

### Example 33

Preparation of Benzyl 3-amino-4-cyano-2-phenyl-2,8-dihydropyrido[3,4-c]pyridazine-7(6H)-carboxylate Hydrochloride (Compound 36)

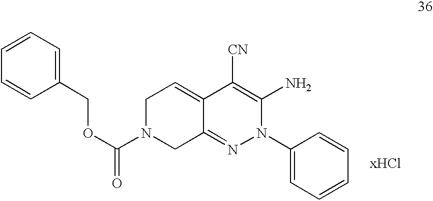

To the hydrazone (0.48 g, 0.0014 mol) in DMSO (0.5 ml) was added malononitrile (0.09 g, 0.0014 mol) and morpholine (0.12 ml, 0.0012 mol) and the reaction mixture stirred at 80° C. for 15 minutes. The mixture was cooled and the product was purified by column chromatography over silica using a gradient of hexane : ethyl acetate (2:1 to ethyl acetate). This gave the pyridazine as a green solid; HPLC (94%); MS (electrospray, [M+H]⁺) m/z 386.5. The HCl salt of the pyridazine was then prepared and the compound freeze-dried to give benzyl 3-amino-4-cyano-2-phenyl-2,8-dihydropyrido[3,4-c]pyridazine-7(6H)-carboxylate hydrochloride (0.32 g, 58%); HPLC (90%, R_{T}=3.38); MS (electrospray, [M+H]⁺) m/z 386.5.

### Example 34

Preparation of 3-amino-2-(2-methoxy-6-methylphenyl)-2,8-dihydro-6H-pyrano[3,4-c]pyridazine-4-carbonitrile Hydrochloride (Compound 37)

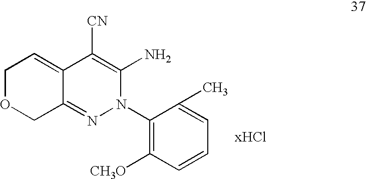

To the hydrazone (0.44 g, 0.0018 mol) in DMSO (1 ml) was added malononitrile (0.117 g, 0.0018 mol) and morpholine (0.16 ml, 0.0018 mol) and the reaction mixture stirred at 80° C. for 15 minutes. The mixture was cooled and the product was purified by column chromatography over silica using a gradient of hexane:ethyl acetate (5:1 to 2:1). This gave the pyridazine as a green solid; HPLC (91%); MS (electrospray, [M+H]⁺) m/z 297.0. The HCl salt of the pyridazine was then prepared and the compound freeze-dried to give 3-amino-2-(2-methoxy-6-methylphenyl)-2,8-dihydro-6H-pyrano[3,4-c]pyridazine-4-carbonitrile hydrochloride (0.29 g, 55%); HPLC (88%, R_{T}=2.98); MS (electrospray, [M+H]⁺) m/z 297.0.

### Example 35

Preparation of 3-amino-2-(3,5-dimethoxyphenyl)-2,8-dihydro-6H-pyrano[3,4-c]pyridazine-4-carbonitrile Hydrochloride (Compound 38)

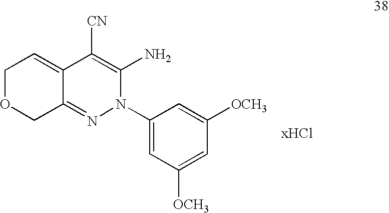

To the hydrazone (0.5 g, 0.0019 mol) in DMSO (0.5 ml) was added malononitrile (0.125 g, 0.0019 mol) and morpholine (0.17 ml, 0.0019 mol) and the reaction mixture stirred at 80° C. for 15 minutes. The mixture was cooled and the product was purified by column chromatography over silica using a gradient of hexane:ethyl acetate (2:1 to ethyl acetate). This gave the pyridazine as a brown solid (0.154 g, 26%); HPLC (93%); MS (electrospray, [M+H]⁺) m/z 313.2. The HCl salt of the pyridazine was then prepared and the compound freeze-dried to give 3-amino-2-(3,5-dimethoxyphenyl)-2,8-dihydro-6H-pyrano[3,4-c]pyridazine-4-carbonitrile hydrochloride; HPLC (95%, R_{T}=3.08); MS (electrospray, [M+H]⁺) m/z 313.2.

### Example 36

Preparation of 3-amino-2-(2-(1-methylpropyl)phenyl)-2,8-dihydro-6H-pyrano[3,4-c]pyridazine-4-carbonitrile Hydrochloride (Compound 39)

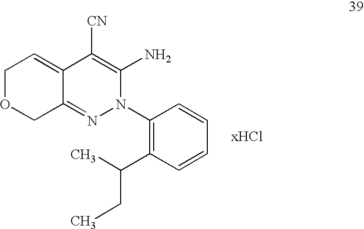

To the hydrazone (0.15 g, 0.00057 mol) in DMSO (1 ml) was added malononitrile (0.038 g, 0.00057 mol) and morpholine (0.05 ml, 0.00057 mol) and the reaction mixture stirred at 80° C. for 15 minutes. The mixture was cooled and the product was purified by column chromatography over silica using a gradient of hexane : ethyl acetate (5:1 to 1:1). This gave the pyridazine as a green solid (0.086 g, 49%); HPLC (89%); MS (electrospray, [M+H]⁺) m/z 309.2. The HCl salt of the pyridazine was then prepared and the compound freeze-dried to give 3-amino-2-(2-(1-methylpropyl)phenyl)-2,8-dihydro-6H-pyrano [3,4-c]pyridazine-4-carbonitrile hydrochloride; HPLC (85%, R_{T}=3.22); MS (electrospray, [M+H]⁺) m/z 309.2.

### Example 37

Preparation of 3-amino-2-(2-chloro-5-methoxyphenyl)-2,8-dihydro-6H-pyrano[3,4-c]pyridazine-4-carbonitrile Hydrochloride (Compound 40)

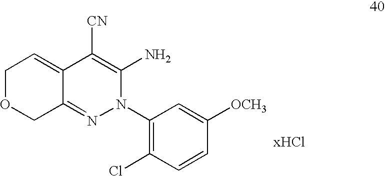

To the hydrazone (0.413 g, 0.00154 mol) in DMSO (1 ml) was added malononitrile (0.102 g, 0.00154 mol) and morpholine (0.13 ml, 0.00154 mol) and the reaction mixture stirred at 80° C. for 15 minutes. The mixture was cooled and the product was purified by column chromatography over silica using a gradient of hexane:ethyl acetate (1:1 to ethyl acetate). This gave the pyridazine as a green solid (0.291 g, 60%); HPLC (96%); MS (electrospray, [M+H]+) m/z 319.2. The HCl salt of the pyridazine was then prepared and the compound freeze-dried to give 3-amino-2-(2-chloro-5-methoxyphenyl)-2,8-dihydro-6H-pyrano[3,4-c]pyridazine-4-carbonitrile hydrochloride; HPLC (93%, R_{T}=2.96); MS (electrospray, [M+H]⁺) m/z 319.2.

### Example 38

Preparation of 3-amino-2-(2-tert-butylphenyl)-2,8-dihydro-6H-pyrano[3,4-c]pyridazine-4-carbonitrile Hydrochloride (Compound 41)

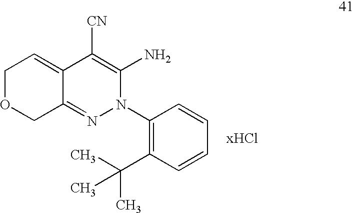

To the hydrazone (1.09 g, 0.0042 mol) in DMSO (1 ml) was added malononitrile (0.28 g, 0.0042 mol) and morpholine (0.37 ml, 0.0042 mol) and the reaction mixture stirred at 80° C. for 15 minutes. The mixture was cooled and the product was purified by column chromatography over silica using a gradient of hexane:ethyl acetate (2:1 to 1:1). This gave the pyridazine as a green solid (0.327 g, 25%); HPLC (96%); MS (electrospray, [M+H]⁺) m/z 309.4. The HCl salt of the pyridazine was then prepared and the compound freeze-dried to give 3-amino-2-(2-tert-butylphenyl)-2,8-dihydro-6H-pyrano[3,4-c]pyridazine-4-carbonitrile hydrochloride; HPLC (91%, R_{T}=3.18); MS (electrospray, [M+H]⁺) m/z 309.4.

### Example 39

Preparation of 3-amino-2-(2-propylphenyl)-2,8-dihydro-6H-pyrano[3,4-c]pyridazine-4-carbonitrile Hydrochloride (Compound 42)

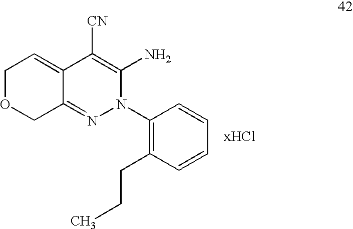

To the hydrazone (0.988 g, 0.00402 mol) in DMSO (1 ml) was added malononitrile (0.27 g, 0.00402 mol) and morpholine (0.35 ml, 0.00402 mol) and the reaction mixture stirred at 80° C. for 15 minutes. The mixture was cooled and the product was purified by column chromatography over silica using a gradient of hexane:ethyl acetate (2:1 to 1:1). This gave the pyridazine as a green solid (0.102 g, 9%); HPLC (91%); MS (electrospray, [M+H]⁺) m/z 295.2. The HCl salt of the pyridazine was then prepared and the compound freeze-dried to give 3-amino-2-(2-propylphenyl)-2,8-dihydro-6H-pyrano[3,4-c]pyridazine-4-carbonitrile hydrochloride; HPLC (91%, R_{T}3.17); MS (electrospray, [M+H]⁺) m/z 295.2.

### Example 40

Preparation of 3-amino-2-[2-(4-morpholinyl)phenyl]-2,8-dihydro-6H-pyrano[3,4-c]pyridazine-4-carbonitrile Hydrochloride (Compound 43)

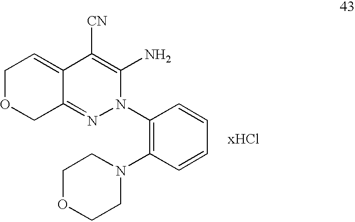

To the hydrazone (0.15 g, 0.00052 mol) in DMSO (1 ml) was added malononitrile (0.035 g, 0.00052 mol) and morpholine (0.045 ml, 0.00052 mol) and the reaction mixture stirred at 80° C. for 15 minutes. The mixture was cooled and the product was purified by column chromatography over silica using a gradient of hexane:ethyl acetate (5:1 to 1:1). This gave the pyridazine as a brown oil (0.08 g, 46%); HPLC (98%); MS (electrospray, [M+H]+) m/z 338.2. The HCl salt of the pyridazine was then prepared and the compound freeze-dried to give BF7550; HPLC (98%, RT=1.41); MS (electrospray, [M+H]+) m/z 338.2.

### Example 41

Preparation of 3-amino-2-(5,6,7,8-tetrahydro-1-naphthalenyl)-2,8-dihydro-6H-pyrano[3,4-c]pyridazine-4-carbonitrile (Compound 44)

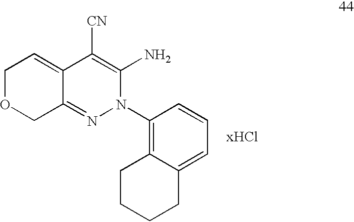

To the hydrazone (0.989 g, 0.00383 mol) in DMSO (1 ml) was added malononitrile (0.253 g, 0.00383 mol) and morpholine (0.33 ml, 0.00383 mol) and the reaction mixture stirred at 80° C. for 15 minutes. The mixture was cooled and the product was purified by column chromatography over silica using a gradient of hexane : ethyl acetate (5:1 to 2:1). This gave the pyridazine as a brown solid (0.472 g, 40%); HPLC (98%); MS (electrospray, [M+H]+) m/z 307.4. The HCl salt of the pyridazine was then prepared and the compound freeze-dried to give 3-amino-2-(5,6,7,8-tetrahydro-1-naphthalenyl)-2,8-dihydro-6H-pyrano[3,4-c]pyridazine-4-carbonitrile; HPLC (98%, RT=1.68); MS (electrospray, [M+H]+) m/z 307.4.

### Example 42

Preparation of 3-amino-2-[1,1′-biphenyl]-2-yl-2,8-dihydro-6H-pyrano[3,4-c]pyridazine-4-carbonitrile (Compound 45)

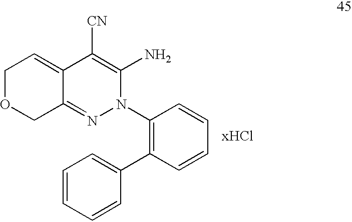

To the hydrazone (0.351 g, 0.00125 mol) in DMSO (0.5 ml) was added malononitrile (0.083 g, 0.00125 mol) and morpholine (0.11 ml, 0.00125 mol) and the reaction mixture stirred at 80° C. for 15 minutes. The mixture was cooled and the product was purified by column chromatography over silica using a gradient of hexane:ethyl acetate (2:1 to ethyl acetate). This gave the pyridazine as a green solid (0.407 g, 91%); HPLC (96%); MS (electrospray, [M+H]+) m/z 329.3. The HCl salt of the pyridazine was then prepared and the compound freeze-dried to give 3-amino-2-[1,1′-biphenyl]-2-yl-2,8-dihydro-6H-pyrano[3,4-c]pyridazine-4-carbonitrile; HPLC (98%, RT=1.75); MS (electrospray, [M+H]+) m/z 329.3.

### Example 43

Preparation of 3-amino-2-[2-(5-methyl-2-furyl)phenyl]-2,8-dihydro-6H-pyrano[3,4-c]pyridazine-4-carbonitrile (Compound 46)

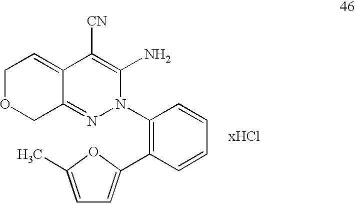

To the hydrazone (0.26 g, 0.000915 mol) in DMSO (0.5 ml) was added malononitrile (0.06 g, 0.000915 mol) and morpholine (0.08 ml, 0.000915 mol) and the reaction mixture stirred at 80° C. for 15 minutes. The mixture was cooled and the product was purified by column chromatography over silica using a gradient of hexane:ethyl acetate (2:1 to 1:1). This gave the pyridazine as a brown solid (0.224 g, 74%); HPLC (99%); MS (electrospray, [M+H]+) m/z 333.3. The HCl salt of the pyridazine was then prepared and the compound freeze-dried to give 3-amino-2-[2-(5-methyl-2-furyl)phenyl]-2,8-dihydro-6H-pyrano[3,4-c]pyridazine-4-carbonitrile; HPLC (99%, RT=1.74); MS (electrospray, [M+H]+) m/z 333.3.

### Example 44

Preparation of 3-amino-2-(2,2,3,3-tetrafluoro-2,3-dihydro-1,4-benzodioxin-5-yl)-2,8-dihydro-6H-pyrano[3,4-c]pyridazine-4-carbonitrile (Compound 47)

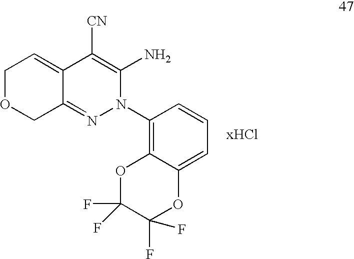

To the hydrazone (0.229 g, 0.000686 mol) in DMSO (0.5 ml) was added malononitrile (0.045 g, 0.000686 mol) and morpholine (0.06 ml, 0.00402 mol) and the reaction mixture stirred at 80° C. for 15 minutes. The mixture was cooled and the product was purified by column chromatography over silica using a gradient of hexane:ethyl acetate (2:1 to ethyl acetate). This gave the pyridazine as a green solid (0.215 g, 82%); HPLC (97%); MS (electrospray, [M+H]+) m/z 383.2. The HCl salt of the pyridazine was then prepared and the compound freeze-dried to give 3-amino-2-(2,2,3,3-tetrafluoro-2,3-dihydro-1,4-benzodioxin-5-yl)-2,8-dihydro-6H-pyrano[3,4-c]pyridazine-4-carbonitrile; HPLC (97%, RT=1.83); MS (electrospray, [M+H]+) m/z 383.2.

### Example 45

Preparation of 3-amino-2-[3-(benzyloxy)phenyl]-2,8-dihydro-6H-pyrano[3,4-c]pyridazine-4-carbonitrile (Compound 48)

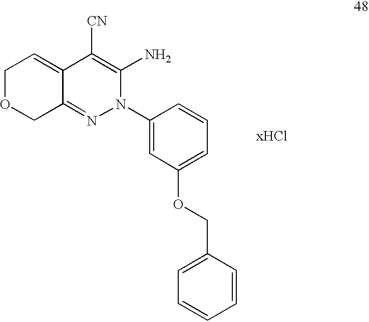

To the hydrazone (0.42 g, 0.00135 mol) in DMSO (1 ml) was added malononitrile (0.09 g, 0.00135 mol) and morpholine (0.12 ml, 0.00135 mol) and the reaction mixture stirred at 80° C. for 15 minutes. The mixture was cooled and the product was purified by column chromatography over silica using a gradient of hexane:ethyl acetate (5:1 to 1:1). This gave the pyridazine as a brown solid (0.27 g, 56%); HPLC (98%); MS (electrospray, [M+H]+) m/z 359.1. The HCl salt of the pyridazine was then prepared and the compound freeze-dried to give 3-amino-2-[3-(benzyloxy)phenyl]-2,8-dihydro-6H-pyrano[3,4-c]pyridazine-4-carbonitrile; HPLC (98%, RT=1.93); MS (electrospray, [M+H]+) m/z 359.1.

### Example 46

Preparation of 3-amino-2-phenyl-2,5,6,8-tetrahydro-3H-pyrano [3,4-c]pyridazine-4-carbonitrile (Compound 49)

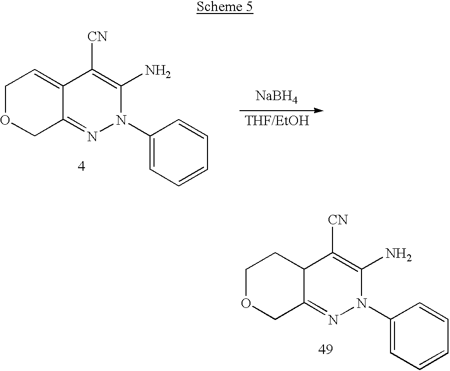

To the pyridazine (4) (0.2 g, 0.0008 mol) in THF/EtOH (2 ml/0.5 ml) was added sodium borohydride (0.06 g, 0.0016 mol) and the reaction mixture stirred at room temperature for 12 hours. The solvent was then removed under reduced pressure, the residue dissolved in DCM, washed with water, dried (MgSO₄) and the solvent removed under reduced pressure, then freeze-dried to give 3-amino-2-phenyl-2,5,6,8-tetrahydro-3H-pyrano[3,4-c]pyridazine-4-carbonitrile (49) as a yellow solid (0.15 g, 74%); HPLC (87%, R_{T}=420); MS (electrospray, [M+H]⁺) m/z 255.3.

### Example 47

Preparation of 3-amino-2-(2-methoxyphenyl)-2,5,6,8-tetrahydro-4aH-pyrano[3,4-c]pyridazine-4-carbonitrile Acetate (Compound 53)

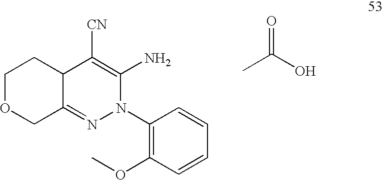

To the pyridazine (31) (0.2 g, 0.0007 mol) in dry dioxan/EtOH (8 ml /2 ml) was added sodium triacetoxyborohydride (0.751 g, 0.00355 mol) and the reaction mixture stirred at 0° C. to room temperature for 12 hours according to the method described in Scheme 5. The reaction mixture was quenched by the gradual addition of water then the solvent was removed under reduced pressure. The residue was dissolved in DCM, washed with saturated sodium bicarbonate solution, water, dried (MgSO₄) and the solvent removed under reduced pressure, then freeze-dried to give 3-amino-2-(2-methoxyphenyl)-2,5,6,8-tetrahydro-4aH-pyrano[3,4-c]pyridazine-4-carbonitrile acetate (53) as a green solid (0.13 g, 65%); HPLC (91%, R_{T}=2.99); MS (electrospray, [M+H]⁺) m/z 285.1.

### Example 48

Preparation of 3-amino-2-[1,1′-biphenyl]-2-yl-2,5,6,8-tetrahydro-4aH-pyrano[3,4-c]pyridazine-4-carbonitrile Acetate (Compound 54)

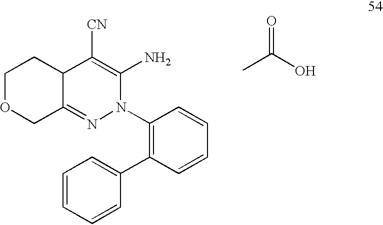

To the pyridazine (45) (0.150 g, 0.00046 mol) in dry dioxan/EtOH (4 ml/0.5 ml) was added sodium triacetoxyborohydride (0.485 g, 0.00229 mol) and the reaction mixture stirred at 0° C. to room temperature for 12 hours according to the method described in Scheme 5. The reaction mixture was quenched by the gradual addition of water then the solvent was removed under reduced pressure. The residue was dissolved in DCM, washed with saturated sodium bicarbonate solution, water, dried (MgSO₄) and the solvent removed under reduced pressure, then freeze-dried to give 3-amino-2-[1,1′-biphenyl]-2-yl-2,5,6,8-tetrahydro-4aH-pyrano[3,4-c]pyridazine-4-carbonitrile acetate (54) as a green solid (0.147 g, 97%); HPLC (89%, R_{T}=3.72); MS (electrospray, [M+H]⁺) m/z 330.6.

### Example 49

Preparation of 3-amino-2-(2,6-dimethylphenyl)-2,5,6,8-tetrahydro-4aH-pyrano[3,4-c]pyridazine-4-carbonitrile Acetate (Compound 55)

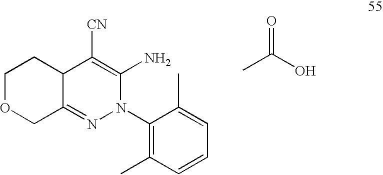

To the pyridazine (14) (0.100 g, 0.00032 mol) in dry dioxan/EtOH (4 ml/0.5 ml) was added sodium triacetoxyborohydride (0.344 g, 0.0016 mol) and the reaction mixture stirred at 0° C. to room temperature for 12 hours according to the method described in Scheme 5. The reaction mixture was quenched by the gradual addition of water then the solvent was removed under reduced pressure. The residue was dissolved in DCM, washed with saturated sodium bicarbonate solution, water, dried (MgSO₄) and the solvent removed under reduced pressure, then freeze-dried to give 3-amino-2-(2,6-dimethylphenyl)-2,5,6,8-tetrahydro-4aH-pyrano[3,4-c]pyridazine-4-carbonitrile acetate ( 55) as a yellow solid (0.07 g, 71%); HPLC (87%, R_{T}=3.66); MS (electrospray, [M+H]⁺) m/z 310.64.

### Example 50

Preparation of 3-amino-2-[4-(trifluoromethyl)phenyl]-2,5,6,8-tetrahydro-4aH-pyrano[3,4-c]pyridazine-4-carbonitrile Acetate (Compound 56)

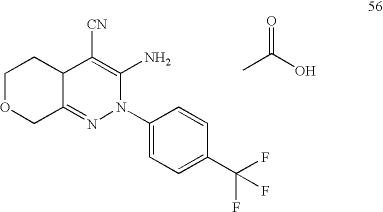

To the corresponding pyridazine (0.02 g, 0.000065 mol) in dry dioxan/EtOH (4 ml 0.5 ml) was added sodium triacetoxyborohydride (0.066 g, 0.00031 mol) and the reaction mixture stirred at 0° C. to room temperature for 12 hours according to the method described in Scheme 5. The reaction mixture was quenched by the gradual addition of water then the solvent was removed under reduced pressure. The residue was dissolved in DCM, washed with saturated sodium bicarbonate solution, water, dried (MgSO₄) and the solvent removed under reduced pressure, then freeze-dried to give 3-amino-2-[4-(trifluoromethyl)phenyl]-2,5,6,8-tetrahydro-4aH-pyrano [3,4-c]pyridazine-4-carbonitrile acetate (56) as a green powder (0.017 g, 81%); HPLC (75%, R_{T}=4.09); MS (electrospray, [M+H]⁺) no m/z.

### Example 51

Preparation of 3-amino-2-(2-methoxyphenyl)-6,6-dimethyl-2,5,6,8-tetrahydro-4aH-pyrano[3,4-c]pyridazine-4-carbonitrile Acetate (Compound 57)

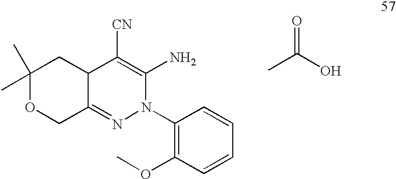

To the corresponding pyridazine (0.100 g, 0.00032 mol) in dry dioxan/EtOH (4 ml/0.5 ml) was added sodium triacetoxyborohydride (0.342 g, 0.0016 mol) and the reaction mixture stirred at 0° C. to room temperature for 12 hours according to the method described in Scheme 5. The reaction mixture was quenched by the gradual addition of water then the solvent was removed under reduced pressure. The residue was dissolved in DCM, washed with saturated sodium bicarbonate solution, water, dried (MgSO₄) and the solvent removed under reduced pressure, then freeze-dried to give 3-amino-2-(2-methoxyphenyl)-6,6-dimethyl-2,5,6,8-tetrahydro-4aH-pyrano[3,4-c]pyridazine-4-carbonitrile acetate (57) as a beige solid (0.029 g, 29%); HPLC (92%, R_{T}=3.31); MS (electrospray, [M+H]⁺) m/z 312.62.

### Example 52

Preparation of 3-amino-2-(3-methoxyphenyl)-2,5,6,8-tetrahydro-4aH-pyrano[3,4-c]pyridazine-4-carbonitrile Acetate (Compound 58)

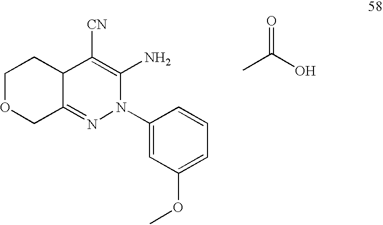

To the pyridazine (13) (0.110 g, 0.00037 mol) in dry dioxan/EtOH (4 ml/0.5 ml) was added sodium triacetoxyborohydride (0.393 g, 0.0018 mol) and the reaction mixture stirred at 0° C. to room temperature for 12 hours according to the method described in Scheme 5. The reaction mixture was quenched by the gradual addition of water then the solvent was removed under reduced pressure. The residue was dissolved in DCM, washed with saturated sodium bicarbonate solution, water, dried (MgSO₄) and the solvent removed under reduced pressure, then freeze-dried to give 3-amino-2-(3-methoxyphenyl)-2,5,6,8-tetrahydro-4aH-pyrano[3,4-c]pyridazine-4-carbonitrile acetate (58) as a yellow powder (0.109 g, 99%); HPLC (84%, R_{T}=3.54); MS (electrospray, [M+H]⁺) m/z 298.59.

### Example 53

Preparation of 3-amino-2-(3-chlorophenyl)-2,5,6,8-tetrahydro-4aH-pyrano[3,4-c]pyridazine-4-carbonitrile Acetate (Compound 59)

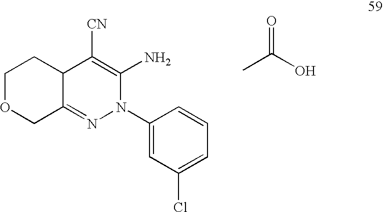

To the corresponding pyridazine (0.100 g, 0.00035 mol) in dry dioxan/EtOH (4 ml 0.5 ml) was added sodium triacetoxyborohydride (0.371 g, 0.0018 mol) and the reaction mixture stirred at 0° C. to room temperature for 12 hours according to the method describe in Scheme 5. The reaction mixture was quenched by the gradual addition of water then the solvent was removed under reduced pressure. The residue was dissolved in DCM, washed with saturated sodium bicarbonate solution, water, dried (MgSO₄) and the solvent removed under reduced pressure, then freeze-dried to give 3-amino-2-(3-chlorophenyl)-2,5,6,8-tetrahydro-4aH-pyrano[3,4-c]pyridazine-4-carbonitrile acetate (59) as a beige solid (0.037 g, 32%); HPLC (100%, R_{T}=3.75); MS (electrospray, [M+H]⁺) m/z 288.5.

### Example 54

Preparation of 3-amino-2-(2-isopropylphenyl)-6,6-dimethyl-2,8-dihydro-6H-pyrano[3,4-c]pyridazine-4-carbonitrile hydrochloride (compound 60)

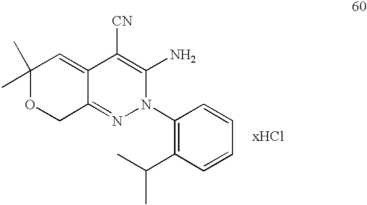

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone (0.256 g, 0.000934 mol) in DMSO(0.5 ml). Malononitrile (0.062 g, 0.000934 mol) and morpholine (0.08 ml, 0.000934 mol) was added and the reaction mixture stirred at 80° C. for 15 minutes. The mixture was cooled and the product was purified by column chromatography over silica using a gradient of hexane:ethyl acetate (3:1 to 2:1). This gave the pyridazine as a yellow oil(0.270 g, 89%); HPLC (98%); MS (electrospray, [M+H]⁺) m/z323.05. The HCl salt of the pyridazine was then prepared and the compound freeze-dried to give 3-amino-2-(2-isopropylphenyl)-6,6-dimethyl-2,8-dihydro-6H-pyrano[3,4-c]pyridazine-4-carbonitrile hydrochloride (60); HPLC (100%, R_{T}=3.34); MS (electrospray, [M+H]+) m/z323.05.

### Example 55

Preparation of 3-amino-2-(2-methoxyphenyl)-6,6-dimethyl-2,8-dihydro-6H-pyrano[3,4-c]pyridazine-4-carbonitrile Hydrochloride (Compound 61)

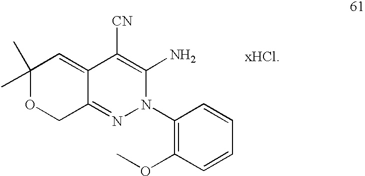

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone(0.360 g, 0.00137 mol) in DMSO(0.5 ml). Malononitrile (0.091 g, 0.00137 mol) and morpholine (0.12 ml, 0.00137 mol) was added and the reaction mixture stirred at 80° C. for 15 minutes. The mixture was cooled and the product was purified by column chromatography over silica using a gradient of hexane: ethyl acetate (5:1 to ethyl acetate). This gave the pyridazine as a yellow solid(0.384 g, 90%); HPLC (90%); MS (electrospray, [M+H]⁺) m/z311.18. The HCl salt of the pyridazine was then prepared and the compound freeze-dried to give 61. HPLC (100%, R_{T}=3.01); MS (electrospray, [M+H]⁺) m/z311.18.

### Example 56

Preparation of 3-amino-2-(2,6-dimethylphenyl)-6,6-dimethyl-2,8-dihydro-6H-pyrano[3,4-c]pyridazine-4-carbonitrile Hydrochloride (Compound 62)

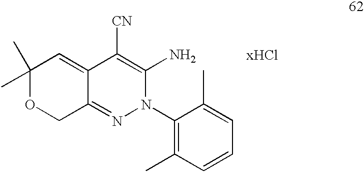

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone(0.350 g, 0.00135 mol) in DMSO(0.5 ml). Malononitrile (0.089 g, 0.00135 mol) and morpholine (0.12 ml, 0.00135 mol) was added and the reaction mixture stirred at 80° C. for 15 minutes. The mixture was cooled and the product was purified by column chromatography over silica using a gradient of hexane: ethyl acetate (5:1 to 1:1). This gave the pyridazine as a yellow solid(0.235 g, 58%); HPLC (96%); MS (electrospray, [M+H]⁺) m/z309.19. The HCl salt of the pyridazine was then prepared and the compound freeze-dried to give 62. HPLC (100%, R_{T}=3.14); MS (electrospray, [M+H]⁺) m/z309.19.

### Example 57

Preparation of 3-amino-2-(3-chlorophenyl)-6,6-dimethyl-2,8-dihydro-6H-pyrano[3,4-c]pyridazine-4-carbonitrile Hydrochloride (Compound 63)

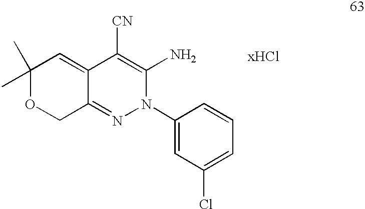

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone (0.344 g, 0.0013 mol) in DMSO (1 ml). Malononitrile (0.085 g, 0.0013 mol) and morpholine (0.11 ml, 0.0013 mol) was added and the reaction mixture stirred at 80° C. for 15 minutes. The mixture was cooled and the product was purified by column chromatography over silica using hexane:ethyl acetate 4:1 as eluant. This gave the pyridazine as a yellow solid (0.373 g, 91%); HPLC (97%); MS (electrospray, [M+H]⁺) m/z 314.56. The HCl salt of the pyridazine was then prepared and the compound freeze-dried to give 63. HPLC (99%, R_{T}=3.14); MS (electrospray, [M+H]⁺) m/z 314.56.

### Example 58

Preparation of 3-amino-2-(3-methoxyphenyl)-6,6-dimethyl-2,8-dihydro-6H-pyrano[3,4-c]pyridazine-4-carbonitrile hydrochloride (compound 64)

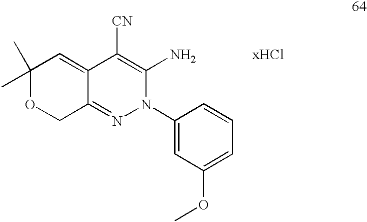

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone (0.47 g, 0.0018 mol) in DMSO (0.5 ml). Malononitrile (0.119 g, 0.0018 mol) and morpholine (0.16 ml, 0.0018 mol) was added and the reaction mixture stirred at 80° C. for 15 minutes. The mixture was cooled and the product was purified by column chromatography over silica using a gradient of hexane: ethyl acetate (4:1 to 1:1) as eluant. This gave the pyridazine as a yellow solid (0.481 g, 86%); HPLC (96%); MS (electrospray, [M+H]⁺) m/z 310.61. The HCl salt of the pyridazine was then prepared and the compound freeze-dried to give 64. HPLC (97%, R_{T}=3.02); MS (electrospray, [M+H]⁺) m/z 310.61.

### Example 59

Preparation of 3-amino-2-(2-methoxy-6-methylphenyl)-6,6-dimethyl-2,8-dihydro-6H-pyrano[3,4-c]pyridazine-4-carbonitrile Hydrochloride (Compound 65)

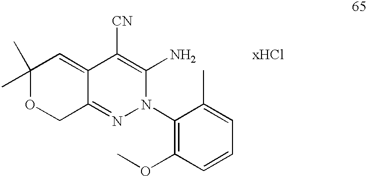

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone (0.291 g, 0.0011 mol) in DMSO (1 ml). Malononitrile (0.070 g, 0.0011 mol) and morpholine (0.09 ml, 0.0011 mol) was added and the reaction mixture stirred at 80° C. for 15 minutes. The mixture was cooled and the product was purified by column chromatography over silica using using a gradient of hexane:ethyl acetate (4:1 to 1:1) as eluant. This gave the pyridazine as a yellow solid (0.379 g, 100%); HPLC (98%); MS (electrospray, [M+H]⁺) m/z 325.20. The HCl salt of the pyridazine was then prepared and the compound freeze-dried to give 65. HPLC (96%, R_{T}=3.03); MS (electrospray, [M+H]⁺) m/z 325.20.

### Example 60

Preparation of 3-amino-6,6-dimethyl-2-[2-(methylsulfanyl)phenyl]-2,8-dihydro-6H-pyrano[3,4-c]pyridazine-4-carbonitrile Hydrochloride (Compound 66)

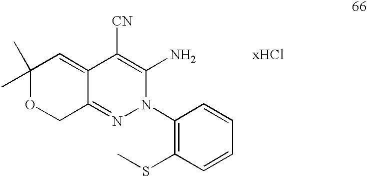

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone (0.291 g, 0.0011 mol) in DMSO (1 ml). Malononitrile (0.084 g, 0.0013 mol) and morpholine (0.11 ml, 0.0013 mol) was added and the reaction mixture stirred at 80° C. for 15 minutes. The mixture was cooled and the product was purified by column chromatography over silica using using a gradient of hexane:ethyl acetate (2:1 to 1:1) as eluant. This gave the pyridazine as a yellow solid (0.361 g, 85%); HPLC (98%); MS (electrospray, [M+H]⁺) m/z 327.16. The HCl salt of the pyridazine was then prepared and the compound freeze-dried to give 66. HPLC (100%, R_{T}=3.13); MS (electrospray, [M+H]⁺) m/z 327.16.

### Example 61

Preparation of 3-amino-6,6-dimethyl-2-[3-isopropylphenyl]-2,8-dihydro-6H-pyrano[3,4-c]pyridazine-4-carbonitrile Hydrochloride (Compound 67)

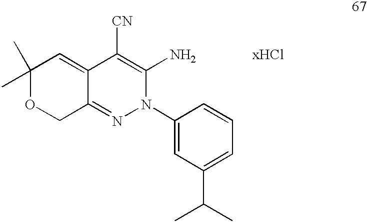

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone (0.39 g, 0.0014 mol) in DMSO (1 ml). Malononitrile (0.094 g, 0.0014 mol) and morpholine (0.12 ml, 0.0014 mol) was added and the reaction mixture stirred at 80° C. for 15 minutes. The mixture was cooled and the product was purified by column chromatography over silica using using a gradient of hexane:ethyl acetate (4:1 to 2:1) as eluant. This gave the pyridazine as a yellow solid (0.226 g, 50%); HPLC (97%); MS (electrospray, [M+H]⁺) m/z 323.21. The HCl salt of the pyridazine was then prepared and the compound freeze-dried to give 67. HPLC (97%, R_{T}=3.39); MS (electrospray, [M+H]⁺) m/z 323.21.

### Example 62

Preparation of 3-amino-6,6-dimethyl-2-[2-phenoxyphenyl]-2,8-dihydro-6H-pyrano[3,4-c]pyridazine-4-carbonitrile hydrochloride (compound 68)

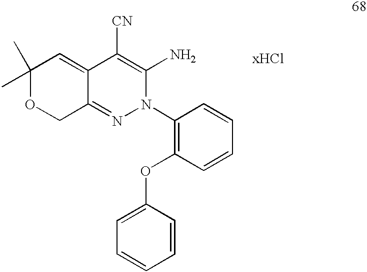

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone (0.197 g, 0.00061 mol) in DMSO (0.5 ml). Malononitrile (0.040 g, 0.00061 mol) and morpholine (0.05 ml, 0.00061 mol) was added and the reaction mixture stirred at 80° C. for 15 minutes. The mixture was cooled and the product was purified by column chromatography over silica using using a gradient of hexane:ethyl acetate (3:1 to 1:1) as eluant. This gave the pyridazine as a yellow solid (0.141 g, 62%); HPLC (98%); MS (electrospray, [M+H]⁺) m/z 373.17. The HCl salt of the pyridazine was then prepared and the compound freeze-dried to give 68. HPLC (98%, R_{T}=3.41); MS (electrospray, [M+H]⁺) m/z 373.17.

### Example 63

Preparation of 3-amino-6,6-dimethyl-2-[2-chlorophenyl]-2,8-dihydro-6H-pyrano[3,4-c]pyridazine-4-carbonitrile Hydrochloride (Compound 69)

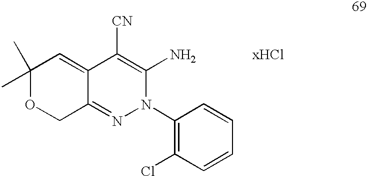

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone (0.320 g, 0.0012 mol) in DMSO (1 ml). Malononitrile (0.080 g, 0.0012 mol) and morpholine (0.11 ml, 0.0012 mol) was added and the reaction mixture stirred at 80° C. for 15 minutes. The mixture was cooled and the product was purified by column chromatography over silica using a gradient of hexane:ethyl acetate (5:1 to 2:1) as eluant. This gave the pyridazine as a yellow solid (0.22 g, 58%); HPLC (91%); MS (electrospray, [M+H]⁺) m/z 315.1. The HCl salt of the pyridazine was then prepared and the compound freeze-dried to give 69. HPLC (91% R_{T}=1.70); MS (electrospray, [M+H]⁺) m/z 315.1.

### Example 64

Preparation of 3-amino-6,6-dimethyl-2- [4-fluorophenyl]-2,8-dihydro-6H-pyrano[3,4-c] pyridazine-4-carbonitrile Hydrochloride (Compound 70)

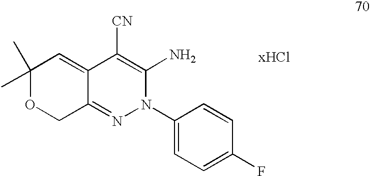

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone (0.190 g, 0.00076 mol) in DMSO (1 ml). Malononitrile (0.050 g, 0.00076 mol) and morpholine (0.07 ml, 0.00076 mol) was added and the reaction mixture stirred at 80° C. for 15 minutes. The mixture was cooled and the product was purified by column chromatography over silica using a gradient of hexane:ethyl acetate (5:1 to 2:1) as eluant. This gave the pyridazine as a yellow solid (0.18 g, 79%); HPLC (96%); MS (electrospray, [M+H]⁺) m/z 299.1. The HCl salt of the pyridazine was then prepared and the compound freeze-dried to give 70. HPLC (96%, R_{T}=1.61); MS (electrospray, [M+H]⁺) m/z 299.1.

### Example 65

Preparation of 3-amino-6,6-dimethyl-2-[4-chlorophenyl]-2,8-dihydro-6H-pyrano[3,4-c]pyridazine-4-carbonitrile Hydrochloride (Compound 71)

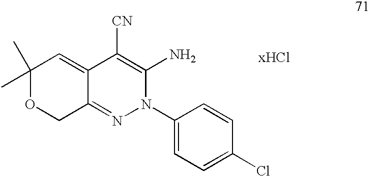

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone (0.381 g, 0.0014 mol) in DMSO (1 ml). Malononitrile (0.094 g, 0.0014 mol) and morpholine (0.12 ml, 0.0014 mol) was added and the reaction mixture was stirred at 80° C. for 15 minutes. The mixture was cooled and the product was purified by column chromatography over silica using hexane:ethyl acetate (2:1) as eluant. This gave the pyridazine as a yellow solid (0.164 g, 37%); HPLC (100%); MS (electrospray, [M+H]⁺) m/z 315.09. The HCl salt of the pyridazine was then prepared and the compound freeze-dried to give 71. HPLC (98%, R_{T}=1.80); MS (electrospray, [M+H]⁺) m/z 315.09.

### Example 66

Preparation of 3-amino-6,6-dimethyl-2-[3-methylphenyl]-2,8-dihydro-6H-pyrano[3,4-c]pyridazine-4-carbonitrile Hydrochloride (Compound 72)

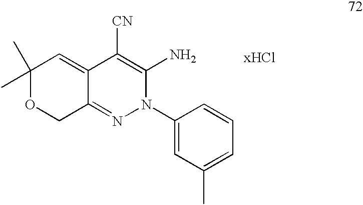

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone (0.253 g, 0.0010 mol) in DMSO (0.5 ml). Malononitrile (0.068 g, 0.0010 mol) and morpholine (0.09 ml, 0.0010 mol) was added and the reaction mixture stirred at 80° C. for 15 minutes. The mixture was cooled and the product was purified by column chromatography over silica using hexane:ethyl acetate (1:1) as eluant. This gave the pyridazine as a yellow solid (0.132 g, 45%); HPLC (98%); MS (electrospray, [M+H]⁺) m/z 295.12. The HCl salt of the pyridazine was then prepared and the compound freeze-dried to give 72. HPLC (98%, R_{T}=1.80); MS (electrospray, [M+H]⁺) m/z 295.12.

### Example 67

Preparation of 3-amino-6,6-dimethyl-2-[3-(dimethylamino)phenyl]-2,8-dihydro-6H-pyrano[3,4-c]pyridazine-4-carbonitrile Hydrochloride (Compound 73)

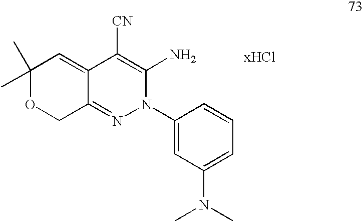

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone (0.138 g, 0.00050 mol) in DMSO (0.5 ml). Malononitrile (0.033 g, 0.00050 mol) and morpholine (0.044 ml, 0.00050 mol) was added and the reaction mixture stirred at 80° C. for 15 minutes. The mixture was cooled and the product was purified by column chromatography over silica using hexane:ethyl acetate (1:1) as eluant. This gave the pyridazine as a yellow solid (0.18 g, 100%); HPLC (99%); MS (electrospray, [M+H]⁺) m/z 324.17. The HCl salt of the pyridazine was then prepared and the compound freeze-dried to give 73. HPLC (98%, R_{T}=1.64); MS (electrospray, [M+H]⁺) m/z 324.17.

### Example 68

Preparation of 3-amino-6,6-dimethyl-2- [2-methylphenyl]-2,8-dihydro-6H-pyrano[3,4-c]pyridazine-4-carbonitrile Hydrochloride (Compound 74)

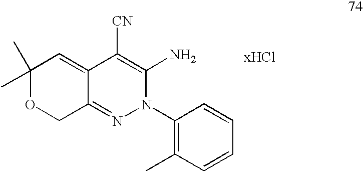

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone (0.259 g, 0.0011 mol) in DMSO (1 ml). Malononitrile (0.070 g, 0.001 mol) and morpholine (0.092 ml, 0.0011 mol) was added and the reaction mixture stirred at 80° C. for 15 minutes. The mixture was cooled and the product was purified by column chromatography over silica using hexane:ethyl acetate (1:1) as eluant. This gave the pyridazine as a yellow solid (0.576 g, 100%); HPLC (100%); MS (electrospray, [M+H]⁺) m/z 295.14. The HCl salt of the pyridazine was then prepared and the compound freeze-dried to give 74. HPLC (100%, R_{T}=1.65); MS (electrospray, [M+H]⁺) m/z 295.14.

### Example 69

Preparation of 3-amino-6,6-dimethyl-2-[3,5-dichlorophenyl]-2,8-dihydro-6H-pyrano[3,4-c]pyridazine-4-carbonitrile Hydrochloride (Compound 75)

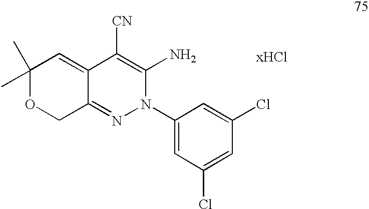

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone (0.349 g, 0.0012 mol) in DMSO (1 ml). Malononitrile (0.077 g, 0.0012 mol) and morpholine (0.101 ml, 0.0012 mol) was added and the reaction mixture stirred at 80° C. for 15 minutes. The mixture was cooled and the product was purified using the Biotage Quad 3 system using a gradient of hexane:ethyl acetate (4:1 to ethyl acetate) as eluant. This gave the pyridazine as a yellow solid (0.108 g, 26%); HPLC (97%); MS (electrospray, [M+H]⁺) m/z 348.95. The HCl salt of the pyridazine was then prepared and the compound freeze-dried to give 75. HPLC (100%, R_{T}=1.93); MS (electrospray, [M+H]⁺) m/z 348.95.

### Example 70

Preparation of 3-amino-6,6-dimethyl-2-[5-chloro-2-methoxyphenyl]-2,8-dihydro-6H-pyrano[3,4-c]pyridazine-4-carbonitrile Hydrochloride (Compound 76)

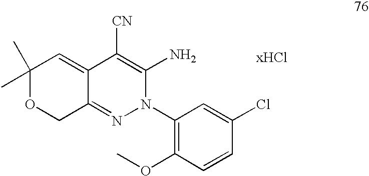

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone (0.489 g, 0.0017 mol) in DMSO (1 ml). Malononitrile (0.109 g, 0.0017 mol) and morpholine (0.144 ml, 0.0017 mol) was added and the reaction mixture stirred at 80° C. for 15 minutes. The mixture was cooled and the product was purified using the Biotage Quad 3 system using a gradient of hexane : ethyl acetate (4:1 to ethyl acetate) as eluant. This gave the pyridazine as a yellow solid (0.406 g, 69%); HPLC (100%); MS (electrospray, [M+H]⁺) m/z 345.01. The HCl salt of the pyridazine was then prepared and the compound freeze-dried to give 76. HPLC (99%, R_{T}=1.87); MS (electrospray, [M+H]⁺) m/z 345.01.

### Example 71

Preparation of 3-amino-6,6-dimethyl-2-[3,5-(dimethoxy)phenyl]-2,8-dihydro-6H-pyrano[3,4-c]pyridazine-4-carbonitrile Hydrochloride (Compound 77)

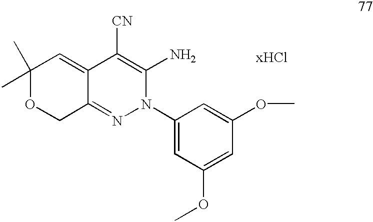

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the the hydrazone (0.304 g, 0.0010 mol) in DMSO (1 ml). Malononitrile (0.069 g, 0.0010 mol) and morpholine (0.09 ml, 0.0010 mol) was added and the reaction mixture stirred at 80° C. for 15 minutes. The mixture was cooled and the product was purified using the Biotage Quad 3 system using a gradient of hexane:ethyl acetate (4:1 to ethyl acetate) as eluant. This gave the pyridazine as a yellow solid (0.234 g, 69%); HPLC (100%); MS (electrospray, [M+H]⁺) m/z 341.04. The HCl salt of the pyridazine was then prepared and the compound freeze-dried to give 77. HPLC (99%, R_{T}=1.83); MS (electrospray, [M+H]⁺) m/z 341.04.

### Example 72

Preparation of 3-amino-6,6-dimethyl-2-[4-chloro-3-(trifluoromethyl)phenyl]-2,8-dihydro-6H-pyrano[3,4-c]pyridazine-4-carbonitrile Hydrochloride (Compound 78)

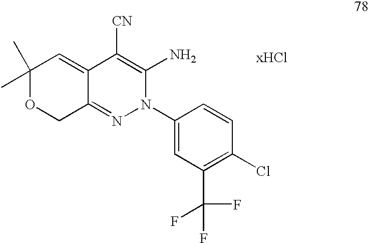

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone (0.290 g, 0.00087 mol) in DMSO (1 ml). Malononitrile (0.057 g, 0.00087 mol) and morpholine (0.08 ml, 0.00087 mol) was added and the reaction mixture stirred at 80° C. for 15 minutes. The mixture was cooled and the product was purified using the Biotage Quad 3 system using a gradient of hexane : ethyl acetate (2:1 to ethyl acetate) as eluant. This gave the pyridazine as a yellow oil (0.17 g, 51%); HPLC (90%); MS (electrospray, [M+H]⁺) m/z 382.9. The HCl salt of the pyridazine was then prepared and the compound freeze-dried to give 78. HPLC (90%, R_{T}=2.09); MS (electrospray, [M+H]⁺) m/z 382.9.

### Example 73

Preparation of 3-amino-6,6-dimethyl-2-[2-(methylsulfonyl)phenyl]-2,8-dihydro-6H-pyrano[3,4-c]pyridazine-4-carbonitrile Hydrochloride (Compound 79)

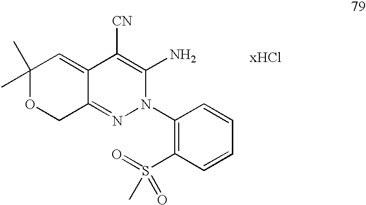

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone (0.220 g, 0.00071 mol) in DMSO (1 ml). Malononitrile (0.047 g, 0.00071 mol) and morpholine (0.06 ml, 0.00071 mol) was added and the reaction mixture stirred at 80° C. for 15 minutes. The mixture was cooled and the product was purified using the Biotage Quad 3 system using a gradient of hexane:ethyl acetate (2:1 to ethyl acetate) as eluant. This gave the pyridazine as a yellow oil (0.15 g, 59%); HPLC (97%); MS (electrospray, [M+H]⁺) m/z 358.9. The HCl salt of the pyridazine was then prepared and the compound freeze-dried to give 79. HPLC (97%, R_{T}=1.50); MS (electrospray, [M+H]⁺) m/z 358.9.

### Example 74

Preparation of 3-amino-6,6-dimethyl-2-[3-nitrophenyl]-2,8-dihydro-6H-pyrano[3,4-c]pyridazine-4-carbonitrile Hydrochloride (Compound 80)

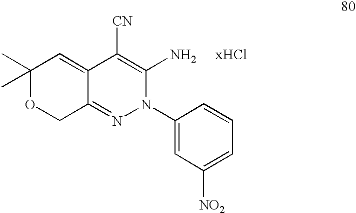

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone (0.300 g, 0.00108 mol) in DMSO (1 ml). Malononitrile (0.072 g, 0.00108 mol) and morpholine (0.094 ml, 0.00108 mol) was added and the reaction mixture stirred at 80° C. for 15 minutes. The mixture was cooled and the product was purified using the Biotage Quad 3 system using a gradient of hexane:ethyl acetate (2:1 to ethyl acetate) as eluant. This gave the pyridazine as an orange solid (0.16 g, 46%); HPLC (98%); MS (electrospray, [M+H]⁺) m/z 326.0. The HCl salt of the pyridazine was then prepared and the compound freeze-dried to give 80. HPLC (98%, R_{T}=1.69); MS (electrospray, [M+H]⁺) m/z 326.0.

### Example 75

Preparation of 3-amino-6,6-dimethyl-2-[3-(methylsulfonyl)phenyl]-2,8-dihydro-6H-pyrano[3,4-c]pyridazine-4-carbonitrile Trifluoroacetate (Compound 81)

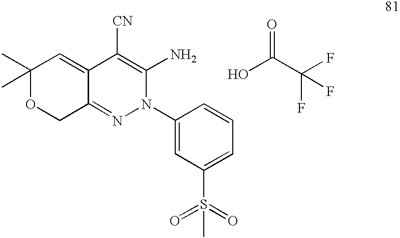

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone (0.240 g, 0.00077 mol) in DMSO (1 ml). Malononitrile (0.051 g, 0.00077 mol) and morpholine (0.07 ml, 0.00077 mol) was added and the reaction mixture stirred at 80° C. for 15 minutes. The mixture was cooled and the product was purified using the Biotage Quad 3 system using a gradient of hexane:ethyl acetate (2:1 to ethyl acetate) as eluant. This gave the pyridazine as a yellow oil (0.10 g, 36%); HPLC (99%); MS (electrospray, [M+H]⁺) m/z 358.7. The HCl salt of the pyridazine was then prepared and the compound freeze-dried to give 81. HPLC (99%, R_{T}=1.55); MS (electrospray, [M+H]⁺) m/z 358.7.

### Example 76

Preparation of 3-amino-6,6-dimethyl-2-[3-fluorophenyl]-2,8-dihydro-6H-pyrano[3,4-c]pyridazine-4-carbonitrile (Compound 82)

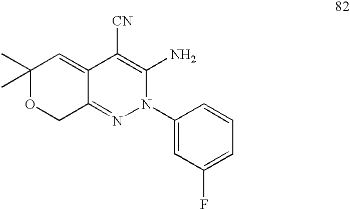

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone (0.57 g, 0.00228 mol) in DMSO (1 ml). Malononitrile (0.151 g, 0.00228 mol) and morpholine (0.2 ml, 0.00228 mol) was added and the reaction mixture stirred at 80° C. for 15 minutes. The mixture was cooled and the product was purified by prep hplc and then freeze-dried to give the TFA salt of the pyridazine as a yellow solid. 82 (0.273 g, 40%); HPLC (100%, R_{T}=1.65); MS (electrospray, [M+H]⁺) m/z 299.1.

### Example 77

Preparation of 3-amino-2-(1,3-benzothiazol-6-yl)-6,6-dimethyl-2,8-dihydro-6H-pyrano[3,4-c]pyridazine-4-carbonitrile trifluoroacetate (compound 83)

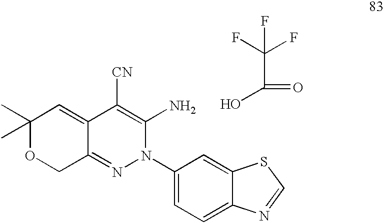

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone (0.30 g, 0.00104 mol) in DMSO (1 ml). Malononitrile (0.069 g, 0.00104 mol) and morpholine (0.09 ml, 0.00104 mol) was added and the reaction mixture stirred at 80° C. for 15 minutes. The mixture was cooled and the product was purified by prep hplc and then freeze-dried to give the TFA salt of the pyridazine as a yellow solid. 83 (0.218 g, 62%); HPLC (100%, R_{T}=1.60); MS (electrospray, [M+H]⁺) m/Z 337.9.

### Example 78

Preparation of 3-amino-2-[4-(1H-imidazol-1-yl)phenyl]-6,6-dimethyl-2,8-dihydro-6H-pyrano[3,4-c]pyridazine-4-carbonitrile Trifluoroacetate (Compound 84)

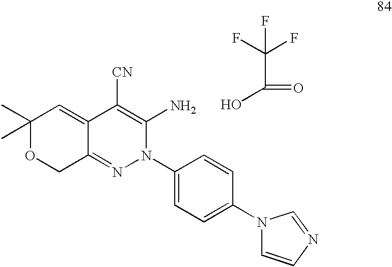

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone (0.201 g, 0.00067 mol) in DMSO (0.5 ml). Malononitrile (0.045 g, 0.00067 mol) and morpholine (0.02 ml, 0.00067 mol) was added and the reaction mixture stirred at 80° C. for 15 minutes. The mixture was cooled and the product was purified by prep hplc and then freeze-dried to give the TFA salt of the pyridazine as a brown oil. 84 (0.162 g, 70%); HPLC (95%, R_{T}=1.22); MS (electrospray, [M+H]⁺) m/z 347.34.

### Example 79

Preparation of 3-amino-6,6-dimethyl-2-[4-(4-morpholinylcarbonyl)phenyl]-2,8-dihydro-6H-pyrano[3,4-c]pyridazine-4-carbonitrile Trifluoroacetate (Compound 85)

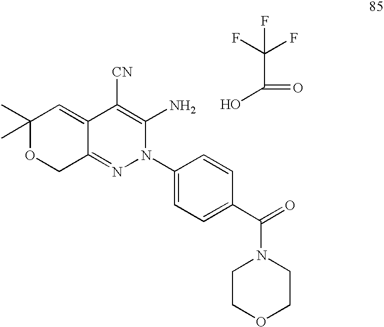

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone (0.437 g, 0.0013 mol) in DMSO (1 ml). Malononitrile (0.084 g, 0.0013 mol) and morpholine (0.11 ml, 0.0013 mol) was added and the reaction mixture stirred at 80° C. for 15 minutes. The mixture was cooled and the product was purified by prep hplc and then freeze-dried to give the TFA salt of the pyridazine as a brown oily-solid. 85 (0.406 g, 79%); HPLC (97%, R_{T}=150); MS (electrospray, [M+H]⁺) m/z 394.07.

### Example 80

Preparation of 3-amino-6,6-dimethyl-2-[2-bromophenyl]-2,8-dihydro-6H-pyrano[3,4-c]pyridazine-4-carbonitrile Trifluoroacetate (Compound 86)

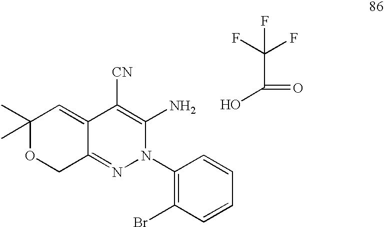

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone (0.306 g, 0.00098 mol) in DMSO (1 ml). Malononitrile (0.065 g, 0.00098 mol) and morpholine (0.086 ml, 0.00098 mol) was added and the reaction mixture stirred at 80° C. for 15 minutes. The mixture was cooled and the product was purified by prep hplc and then freeze-dried to give the TFA salt of the pyridazine as a brown solid. 86 (0.251 g, 71%); HPLC (100%, R_{Y}=1.75); MS (electrospray, [M+H]⁺) m/z 358.74, 361.22.

### Example 81

Preparation of 2-(diethylamino)ethyl 4-(3-amino-4-cyano-6,6-dimethyl-6H-pyrano[3,4-c]pyridazin-2(8H)-yl)benzoate Trifluoroacetate (Compound 87)

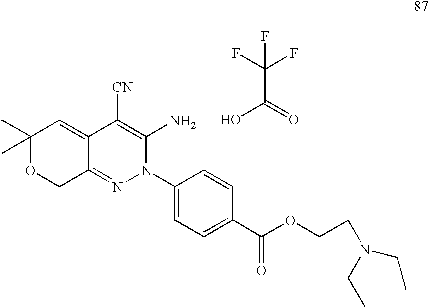

The starting material 2,2-dimethyl-tetrahydro-pyran-4-one (52) was prepared according to method 2, Scheme 3. Synthetic method 1a, Scheme 1, was used to prepare the hydrazone intermediate. The method according to Scheme 4 was undertaken to synthesize the pyridazine using the hydrazone (0.053 g, 0.00014 mol) in DMSO (0.5 ml). Malononitrile (0.0093 g, 0.00014 mol) and morpholine (0.012 ml, 0.00014 mol) was added and the reaction mixture stirred at 80° C. for 15 minutes. The mixture was cooled and the product was purified by prep hplc and then freeze-dried to give the TFA salt of the pyridazine as a green oil. 87 (0.038 g, 64%); HPLC (100%, R_{T}=1.45); MS (electrospray, [M+H]⁺) m/z 424.07.

### Example 82

Preparation of 3-amino-6,6-dimethyl-2-[2-(trifluoromethyl)phenyl]-2,8-dihydro-6H-pyrano[3,4-clpyridazine-4-carbonitrile Trifluoroacetate (Compound 89)

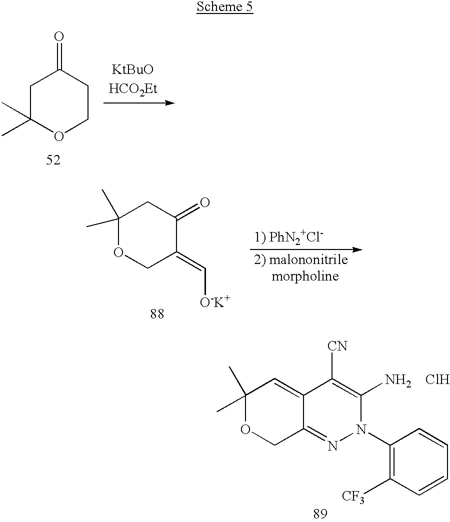

The starting material 2,2-dimethyl-tetrahydro-pyran-4-one (52) was prepared according to method 2, Scheme 3. A solution of 2,2-dimethyl-tetrahydro-pyran-4-one (3.00 g, 23.4 mmol) and ethyl formate (3.0 ml, 37.2 mmol) in dry THF (30 mL) was placed under nitrogen. To the solution was added K^{t}BuO (2.63 g, 23.4 mmol) in small portions during 10 min. A yellow precipitate was formed almost immediately. The mixture was stirred at room temperature for 3 hours. The solvent was then evaporated and the crude product 88 was dried in vacuum. The crude material was dissolved in EtOH (18 mL) and divided into 6 portions and stored in freezer until used.

A solution of the diazoniumsalt of 2-trifluoromethyl-aniline was prepared by the gradual addition of a solution of sodium nitrite (273 mg, 3.95 mmol) in water (2 mL) to a solution of 2-trifluoromethyl aniline (637 mg, 3.95 mol) in 1.00 M HCl (7.90 mL, 7.90 mmol) at −5° C. The cold ethanol solution (3 mL) of the the potassium salt of 5-(hydroxymethylene)-2,2-dimethyltetrahydro-4H-pyran-4-one 88 (3.90 mmol) was added dropwise and the resulting mixture was stirred vigorously at −5° C. for 1 h. The reaction mixture was diluted with DCM and washed with water. The organic phase was dried with MgSO4 and evaporated and gave 0.9755 g of the hydrazone intermediate as an orange solid. (83%) ¹H NMR (400 MHz, CDCl₃) δ1.36 (s, 6 H), 2.58 (s, 2 H), 4.64 (s, 2 H), 7.08 (t, 1 H), 7.50 (t, 1 H), 7.55 (d, 1 H), 7.86 (d, 1 H), 14.05 (s, 0.7 H). MS (EI) m/z 300.0 (M⁺). A solution of hydrazone (0.97 g, 3.23 mmol), malononitrile (261 mg, 3.95 mmol) and morpholine (345 μl, 3.95 mmol) in DMSO (2 ml) was heated at 80° C. 30 min. The reaction mixture was diluted with CHCl₃, was washed with water, dried (MgSO₄) and evaporated. The crude product was purified by column chromatography on silica gel with a gradient of hexane : ethyl acetate (4:1 to 1:1). The purified material was dissolved in EtOAc and HCl in EtOAc was added. The product was filtered, washed with EtOAc and dried in vacuum. Gave 0.667 g of the title compound 89. (54%) MS (ESI+) m/z 349.29 (M+H)⁺. HPLC (30-60%) rₜ:12.46 min (99%).

### Example 83

Preparation of 3-amino-6,6-dimethyl-2-13-(trifluoromethyl)phenyl]-2,8-dihydro-6H-pyrano[3,4-c]pyridazine-4-carbonitrile Hydrochloride (Compound 90)

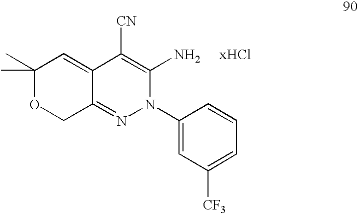

The intermediate 6,6-dimethyldihydro-2H-pyran-3,4-3-{[3-(trifluoromethyl)-phenyl]hydrazone} was synthesized according to the modified method 1c described in example 82, Scheme 5. The hydrazone (0.948 g, 3.16 mmol) was dissolved in DMSO (2 mL) and malononitrile (263 mg, 3.98 mmol) and morpholine (345 μL, 3.96 mmol) was added. The reaction conditions and work-up procedure followed Method 2a and gave 0.514 g of the title compound 90. (42%) MS (ESI+) m/z 349.31 (M+H)⁺. HPLC (30-60%) rₜ:13.75 min (99%)

### Example 84

Preparation of 3-amino-2-(4-methoxyphenyl)-6,6-dimethyl-2,8-dihydro-6H-pyrano[3,4-c]pyridazine-4-carbonitrile Hydrochloride (Compound 91)

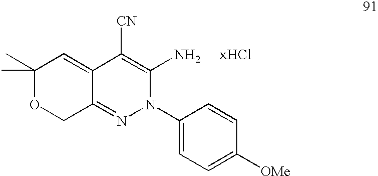

The intermediate 6,6-dimethyldihydro-2H-pyran-3,4-dione 3-[(4-methoxy-phenyl)hydrazone] was synthesized according to the modified method 1c described in example 82, Scheme 5. The hydrazone (0.832 g, 3.17 mmol) was dissolved in DMSO (2 mL) and malononitrile (265 mg, 4.01 mmol) and morpholine (345 μL, 3.96 mmol) was added. The reaction conditions and work-up procedure followed Method 2a and gave 0.651 g of the title compound 91. (59%) MS (ESI+) m/z 311.31 (M+H)⁺.HPLC (30-60%) rₜ:9.74 min

### Example 85

Preparation of 3-amino-2-(2-fluorophenyl)-6,6-dimethyl-2,8-dihydro-6H-pyrano[3,4-c]pyridazine-4-carbonitrile Hydrochloride (Compound 92)

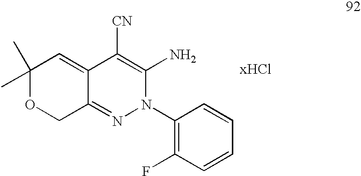

The intermediate 6,6-dimethyldihydro-2H-pyran-3,4-dione 3-[(2-fluorophenyl)hydrazone] was synthesized according to the modified method 1c described in example 82, Scheme 5. The hydrazone (0.778 g, 3.16 mmol) was dissolved in DMSO (2 mL) and malononitrile (268 mg, 4.06 mmol) and morpholine (345 μL, 3.96 mmol) was added. The reaction conditions and work-up procedure followed Method 2a and gave 0.625 g of the title compound 92. (60%) MS (ESI+) m/z 299.30 (M+H)⁺. HPLC (30-60%) rₜ:8 45 min (99%)

### Example 86

Preparation of 3-amino-2-[4-(2-hydroxyethyl)phenyl]-6,6-dimethyl-2,8-dihydro-6H-pyrano[3,4-c]pyridazine-4-carbonitrile Hydrochloride (Compound 93)

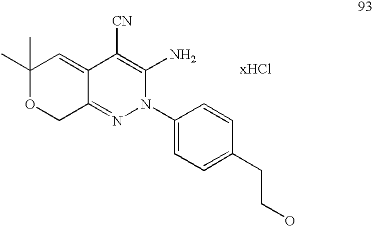

The intermediate 6,6-dimethyldihydro-2H-pyran-3,4-dione 3-{[4-(2-hydroxyethyl)-phenyl]hydrazone} was synthesized according to the modified method 1c described in example 82, Scheme 5. The hydrazone (0.832 g, 3.01 mmol) was dissolved in DMSO (2 mL) and malononitrile (266 mg, 4.03 mmol) and morpholine (345 μL, 3.96 mmol) was added. The reaction conditions and work-up procedure followed Method 2a and gave 0.690 g of the title compound 93. (64%) MS (ESI+) m/z 325.33 (M+H)⁺. HPLC (30-60%) rₜ:5.88 min (97%)

### Example 87

Preparation of 3-amino-2-[3-(hydroxymethyl)phenyl]-6,6-dimethyl-2,8-dihydro-6H-pyrano[3,4-c]pyridazine-4-carbonitrile Hydrochloride (Compound 94)

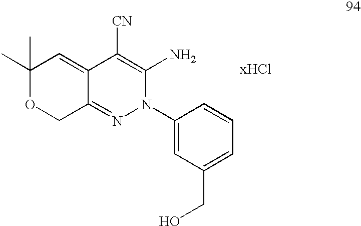

The intermediate 6,6-dimethyldihydro-2H-pyran-3,4-dione 3-{[3-(hydroxymethyl)phenyl]-hydrazone} was synthesized according to the modified method 1c described in example 82, Scheme 5. The hydrazone (0.546 g, 2.08 mmol) was dissolved in DMSO (2 mL) and malononitrile (170 mg, 2.57 mmol) and piperidine (255 μL, 2.58 mmol) was added. The reaction conditions and work-up procedure followed Method 2a and gave 0.538 g of the title compound 94 (75%) MS (ESI+) m/z 311.26(M+H)⁺. HPLC (30-60%) rₜ:5.35 (94%).

### Example 88

Preparation of 3-amino-2-(4-ethylphenyl)-6,6-dimethyl-2,8-dihydro-6H-pyrano[3,4-c]pyridazine-4-carbonitrile Hydrochloride (Compound 95)

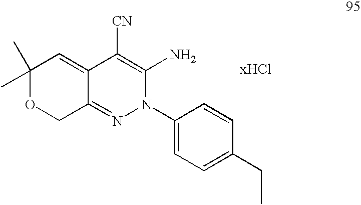

The intermediate 6,6-dimethyldihydro-2H-pyran-3,4-dione 3-[(4-ethylphenyl)hydrazone] was synthesized according to the modified method 1c described in example 82, Scheme 5. The hydrazone (0.526 g, 2.02 mmol) was dissolved in DMSO (2 mL) and malononitrile (170 mg, 2.57 mmol) and piperidine (255 μL, 2.58 mmol) was added. The resulting mixture was heated at 80° C. for 30 min. The reaction mixture was diluted with CHCl₃, washed with water, dried (MgSO₄) and evaporated. The crude product was dissolved in EtOAc/Et₂O and HCl in EtOAc was added dropwise. The product was filtered, washed with EtOAc and Et₂O and dried in vacuum and gave 0.488 g of the title product 95. (70%) MS (ESI+) m/z 309 (M+H)⁺. HPLC (30-60%) rₜ:13.46 min (99%)

### Example 89

Preparation of 3-amino-2-[(3-carboxymethyl)phenyl]-6,6-dimethyl-2,8-dihydro-6H-pyrano[3,4-c]pyrazidine-4-carbonitrile Hydrochloride (Compound 96)

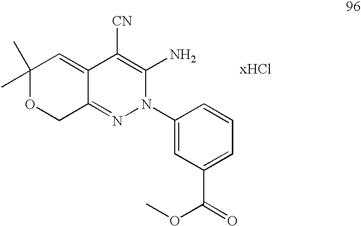

The intermediate 6,6-dimethyldihydro-2H-pyran-3,4-dione [3-(3-carboxymethylphenyl)-hydrazone] was synthesized according to the modified method 1c described in example 82, Scheme 5. The hydrazone (0.461 g, 1.59 mmol) was dissolved in DMSO (2 mL) and malononitrile (126 mg, 1.91 mmol) and morpholine (170 μL, 1.95 mmol) was added. The reaction mixture was stirred at room temperature for 1 h. The reaction mixture was diluted with EtOAc and washed with water, then extracted with 1 M HCl (aq.)×3. The aqueous phase was neutralized and the free amine was back-extracted to EtOAc. The organic phase was dried (MgSO₄) and evaporated. The pure amine was redissolved in EtOAc/Et₂O and HCl (Et₂O) was added. The product was filtered, washed with Et₂O and dried in vacuum and gave 0.281 g of the title compound 96. (47%) MS (ESI+) for m/z 339.23 (M+H)^{+. HPLC (}30-60% in 10 min) rₜ:8.36 min (96%)

### Example 90

Preparation of 3-amino-2-(3-acetamidophenyl)-6,6-dimethyl-2,8-dihydro-6H-pyrano [3,4-c]pyrazidine-4-carbonitrile Hydrochloride (Compound 97)

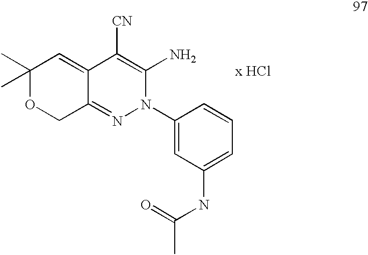

The intermediate 6,6-dimethyldihydro-2H-pyran-3,4-dione [3-(3-acetamidophenyl)-hydrazone] was synthesized according to the modified method 1c described in example 82, Scheme 5. The hydrazone (0.481 g, 1.66 mmol) was dissolved in DMSO (2 mL) and malononitrile (133 mg, 2.01 mmol) and morpholine (170 μL, 1.95 mmol) was added. The reaction conditions and work-up procedure followed the method described in Example 89 and gave 0.337 g of the title compound 97. (54%) MS (ESI+) m/z 338.26 (M+H)⁺. HPLC (30-60%) rₜ:6.13 min (97%)

### Example 91

Preparation of 3-amino-6,6-dimethyl-2-(2-nitrophenyl)-2,8-dihydro-6H-pyrano[3,4-c]pyridazine-4-carbonitrile Hydrochloride (Compound 98)

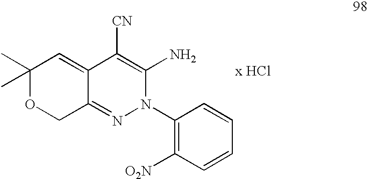

The intermediate 6,6-dimethyldihydro-2H-pyran-3,4-dione 3-[(2-nitrophenyl)-hydrazone]was synthesized according to the modified method 1c described in example 82, Scheme 5. The hydrazone (0.443 g, 1.59 mmol) was dissolved in DMSO (2 mL) and malononitrile (145 mg, 2.19 mmol) and morpholine (170 μL, 1.95 mmol) was added. The reaction conditions and work-up procedure followed the method described in Example 89 and gave 0.348 g of the title compound 98. (61%). MS (ESI+) m/z 326.23 (M+H)⁺. HPLC (30-60%) rₜ:9.58 min (97%)

### Example 92

Preparation of 3-amino-2-(2-butylphenyl)-6,6-dimethyl-2,8-dihydro-6H-pyrano[3,4-c]pyridazine-4-carbonitrile Hydrochloride (Compound 99)

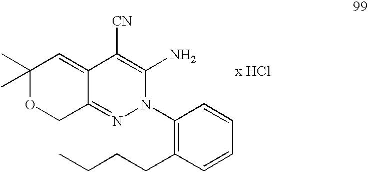

The intermediate 6,6-dimethyldihydro-2H-pyran-3,4-dione 3-[(2-butylphenyl)hydrazone] was synthesized according to the modified method 1c described in example 82, Scheme 5. The hydrazone (0.477 g, 1.65 mmol) was dissolved in DMSO (2 mL) and malononitrile (131 mg, 1.98 mmol) and morpholine (170 μL, 1.95 mmol) was added. The reaction conditions and work-up procedure followed the method described in Example 89 and gave 0.339 g of the title compound 99. (55%). MS (ESI+) m/z 337.29 (M+H)⁺. HPLC (60-90%) rt:5.39 min (94%).

### Example 93

Preparation of 3-amino-6,6-dimethyl-2-(3-phenoxyphenyl)-2,8-dihydro-6H-pyrano[3,4-c]pyridazine-4-carbonitrile Hydrochloride (Compound 100)

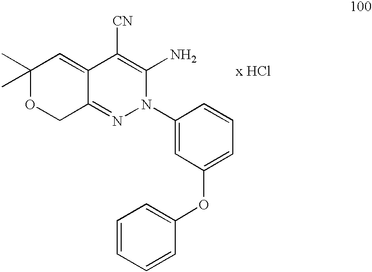

The intermediate 6,6-dimethyldihydro-2H-pyran-3,4-dione 3-[(3-phenoxyphenyl)-hydrazone] was synthesized according to the modified method 1c described in example 82, Scheme 5. The hydrazone (0.579 g, 1.78 mmol) was dissolved in DMSO (2 mL) and malononitrile (132 mg, 2.00 mmol) and morpholine (170 μL, 1.95 mmol) was added. The reaction conditions and work-up procedure followed the method described in Example 89 and gave 0.367 g of the title compound 100. (50%) MS (ESI+) e m/z 373.23 (M+H)⁺. HPLC (60-90%) rₜ:4.99 min (94%).

### Example 94

Preparation of 3-amino-6,6-dimethyl-2-[2-(4-morpholinyl)phenyl]-2,8-dihydro-6H-pyrano[3,4-c]pyridazine-4-carbonitrile Hydrochloride (Compound 101)

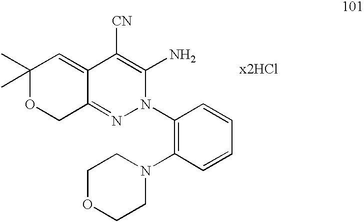

The intermediate 6,6-dimethyldihydro-2H-pyran-3,4-dione 3-{[2-(4-morpholinyl)phenyl]-hydrazone} was synthesized according to the modified method 1c described in example 82, Scheme 5, with the addition of an extra equivalent of acid when forming the diazonium salt. The hydrazone (0.428 g, 1.35 mmol) was dissolved in DMSO (10 mL) and malononitrile (187 mg, 2.83 mmol) and piperazinomethyl polystyrene resin (1.30 g, 1.08 mmol/g, 1 eq.) was added. The reaction mixture was heated at 80° C. for 8 h, then diluted with CH₂Cl₂ and the solid phase reagent was filtered and washed with CH₂Cl₂. The organic phase was washed with water, dried (MgSO₄) and evaporated. The crude product was purified by column chromatography on silica gel with petroleum ether:EtOAc 4:1. The purified material was dissolved in EtOAc/Et₂O and HCl in Et₂O was added dropwise. The product was filtered, washed with Et₂O and dried in vacuum and gave 0.302 g of the title compound 101. (51%) MS (ESI+) m/z 366.28 (M+H)⁺. HPLC (30-60%) rₜ:10.58 min (98%).

### Example 95

Preparation of 3-amino-2-(2-ethoxyphenyl)-6,6-dimethyl-2,8-dihydro-6H-pyrano[3,4-c]pyridazine-4-carbonitrile Hydrochloride (Compound 102)

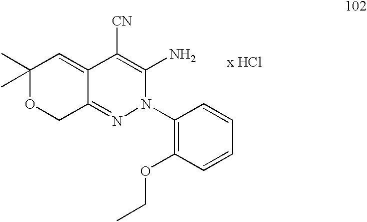

The intermediate 6,6-dimethyldihydro-2H-pyran-3,4-dione 3-[(2-ethoxyphenyl)hydrazone] was synthesized according to the modified method 1c described in example 82, Scheme 5. The hydrazone (0.491 g, 1.78 mmol) was dissolved in DMSO (10 mL) and malononitrile (289 mg, 4.37 mmol) and piperazinomethyl polystyrene resin (1.67 g, 1.08 mmol/g, 1 eq.) was added. The reaction mixture was heated at 80° C. for 12h. The reaction mixture was diluted with CH₂Cl₂ and the resin was filtered and washed with CH₂Cl₂. The organic phase was washed with water and extracted with 1 M HCl (aq.)×3. The aqueous phase was neutralized and the free amine was back-extracted with EtOAc, dried (MgSO₄) and evaporated. The pure product was dissolved in Et₂O and HCl in Et₂O was added dropwise. The product was filtered, washed with Et₂O and dried in vacuum and gave 0.349 g of the title compound 102. (54%) MS (ESI+) m/z 325.29 (M+H)⁺. HPLC (30-60%) rₜ:12.23 min (98%).

Preparation of the Intermediate Monomethyl Pyranone According to Method 2:

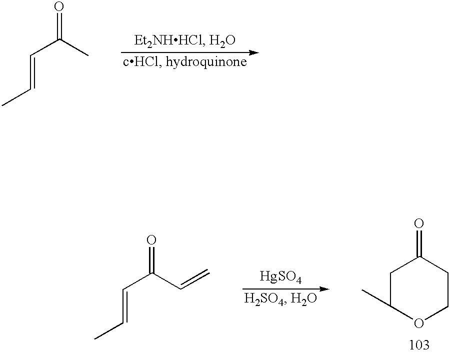

Diethylaminehydrochloride(24.87 g, 0.24 mol), formaldehyde solution (19.51 g, 0.24 mol), 3-penten-2-one (23.2 ml, 0.24 mol), concentrated HCl(1 ml; for pH=1) and hydroquinone (0.53 g) were heated at 80° C. for two hours. The reaction mixture was then distilled under reduced pressure. The divinyl ketone was collected at P=16 mmHg, Tᵥₐₚ=45-70° C. (8.08 g, 35% yield).

Divinyl ketone (8.0 g, 0.083 mol) was added dropwise to a mixture of HgSO₄(0.616 g, 0.0027 mol), H₂SO₄(0.616 ml) and water (24 ml) at 85° C. with stirring. Then HgSO₄ (0.152 g, 0.00052 mol) and H₂SO₄ (0.152 ml) in water (6 ml) were added over a period of one hour. In total the reaction mixture was heated for five hours at 85 to 100° C. This reaction mixture was then steam distilled. The product was then extracted with DCM, washed with water, dried (MgSO₄) and the solvent removed under reduced pressure to give a yellow liquid. This crude product 103 (80%) was then used to synthesize the monomethyl analogues. The pyranone (colourless liquid) had a yield of 2.38 g, 18%. NMR: δ1.29 (d, 3H), δ2.34 (d, 2H), δ2.36 (t, 2H), δ3.77 (t, 2H), δ4.22 (q, 1H).

### Example 96

Preparation of 3-amino-6-methyl-2,8-dihydro-6H-pyrano[3,4-c]pyridazine-4-carbonitrile Trifluoroacetate (Compound 104)

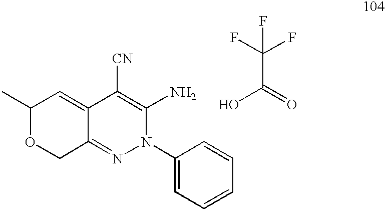

To the monomethyl hydrazone 103 (0.166 g, 0.00076 mol) in DMSO (0.5 ml) was added malononitrile (0.050 g, 0.00076 mol) and morpholine (0.07 ml, 0.00076 mol) and the reaction mixture stirred at 80° C. for 15 minutes. The mixture was cooled and the product was purified by prep HPLC and then freeze-dried to give the TFA salt of the pyridazine as a green-brown solid, 104 (0.07 g, 35%); HPLC (99%, R_{T}=1.42); MS (electrospray, [M+H]⁺) m/z266.99.

### Example 97

Preparation of 3-amino-2-(2-ethoxyphenyl)-6-methyl-2,8-dihydro-6H-pyrano [3,4-c]pyridazine-4-carbonitrile Trifluoroacetate (Compound 105)

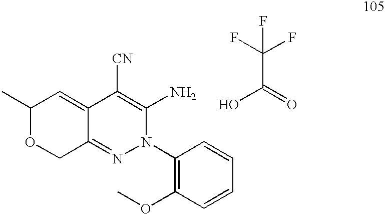

To the monomethyl hydrazone 103 (0.248 g, 0.001 mol) in DMSO (1.0 ml) was added malononitrile (0.05 g, 0.001 mol) and morpholine (0.06 ml, 0.001 mol) and the reaction mixture stirred at 80° C. for 15 minutes. The mixture was cooled and the product was purified by prep HPLC and then freeze-dried to give the TFA salt of the pyridazine as a green oil, 105 (0.252 g, 85%); HPLC (99%, R_{T}1.54); MS (electrospray, [M+H]⁺) m/z297.12

### Pharmaceutical Compositions

The novel compounds according to the present invention may be administered orally, intranasally, transdermally, subcutaneously, parenterally, intramusculary, as well as intravenously. Oral administration is the preferred route.

The dosage will depend on the route of administration, the severity of the disease, age and weight of the patient, and other factors normally considered by the attending physician when determining the individual regimen and dosage level as the most appropriate for a particular patient.

Either solid or fluid dosage forms can be prepared for oral administration. Solid compositions, such as compressed tablets, are prepared by mixing the compounds of the invention with conventional ingredients such as talc, magnesium stearate, dicalcium phosphate, magnesium aluminum silicate, calcium sulfate, starch, lactose, acacia, methyl cellulose, or functionally similar pharmaceutical diluents and carriers. Capsules are prepared by mixing the compounds of this invention with an inert pharmaceutical diluent and placing the mixture into an appropriately sized hard gelatin capsule. Soft gelatin capsules are prepared by machine encapsulation of a slurry of the compounds of this invention with an acceptable inert oil such as vegetable oil or light liquid petrolatum.

Syrups are prepared by dissolving compounds of the invention in an aqueous vehicle and adding sugar, aromatic flavoring agents and preservatives. Elixirs are prepared using a hydroalcoholic vehicle such as ethanol, suitable sweeteners such as sugar or saccharin and an aromatic flavoring agent. Suspensions are prepared with an aqueous vehicle and a suspending agent such as acacia, tragacanth, or methyl cellulose.

When the compounds of the invention are administered parenterally, they can be given by injection or by intravenous infusion. Parenteral solutions are prepared by dissolving the compounds of the invention in aqueous vehicle and filter sterilizing the solution before placing in a suitable sealable vial or ampule. Parenteral suspensions are prepared in substantially the same way except a sterile suspension vehicle is used and the compounds of the present invention are sterilized with ethylene oxide or suitable gas before it is suspended in the vehicle.

Thus, a further aspect of the present invention is a pharmaceutical composition comprising a compound of formula I, Ia, Ia′, and Ib (e.g., a compound specified in the following Examples) above respectively together with a pharmacologically and pharmaceutically acceptable carrier. It is preferred to use pharmaceutically inert carriers which may be solid or liquid. Solid form preparations include but is not limited to powders, tablets, dispersible granules, capsules etc. The skilled person within the formulation field will readily know which carrier to use for the specific circumstance when formulating a composition in accordance with the present invention.

Pharmaceutically acceptable salts of the compounds of formula I above, which salts are useful in accordance with the present invention, may be formed from organic and inorganic acids. Examples of such salts are hydrochloride salts, tosylate salts, citrate salts, maleate salts, acetate salts, hydrobromide salts, malate salts, stearate salts, aluminium salts, lithium salts, calcium salts, and magnesium salts among others. This list should however not in any way be regarded as exhaustive. The hydrochloride salts are the preferred salts of the invention.

### Biological Evaluation

The compound 6H-Pyrano[3,4-c]pyridazine-4-carbonitrile, 3-amino-2,8-dihydro-6,6-dimethyl-2-phenyl which is commercially available from the company Labotest in Germany, was tested for biological activity in accordance with the present invention, together with other compounds within the scope of the present invention.

Expression and Purification of Recombinant Human PTPs

Human PTP1B (amino acid residues 1-298, cloned from a human placental library), without the GST tag and thrombin cleavage site, was inserted into a pMB replicon and transformed into E. coli BL21 (DE3), a strain containing a chromosomal copy of the gene for T7 RNA polymerase under control of a lacUV5 promoter. Expression of PTP1B was induced with isopropyl thiogalactose and cells were lysed in lysis buffer comprising 50 mM Tris-HCl pH 7.5, 10% glycerol, 1 mM EDTA, 3 mM DTT, 3 mM MgCl₂, and 0.2 mg/ml lysozyme with 1 mg/ml DNAse I. The soluble protein was purified by ion exchange, hydrophobic interaction and gel exclusion chromatography for use in assays to identify PTP1B inhibitors. The plasmid pGEX2K-SHP2 which encoded the catalytic domain of human SHP-2 (residues 252-529) was used to transform E. coli cells. After induction of protein expression, cells were lysed in PBS containing 1% Triton X100 and lysozyme (2 mg/ml). Recombinant protein was purified by glutathione sepharose 4B chromatography followed by Superdex 200 size exclusion chromatography. Recombinant proteins were stored at −70° C. until used. Recombinant T cell PTP (TCPTP) and LAR were purchased from New England Biolabs.

Measurement of PTP Activity

Human PTP1B activity was measured using p-nitrophenol phosphate (pNPP) as substrate in a 96-well microtiter plate format. An assay pH of 7.2 is used for standard assays (measured extinction coefficient =9800 at pH 7.2).

Standard assays were conducted at room temperature in a total volume of 0.2 ml that contains Hepes buffer (50 mM, pH 7.2), NaCl (50 mM), EDTA (1 mM), DTT (1 mM), bovine serum albumin (1 mg/ml), pNPP (1.25 mM) and PTP1B (500 ng/ml, 13.5 nM). A master plate was set up for each compound in which a stock solution of compound in DMSO (19 mM or 1 mM) was diluted 1 to 10 with assay buffer in column 1 (giving a 1 mM or 100 μM concentration). Substances were subsequently diluted serially by two thirds in all columns across the plate. For enzyme assays, 20 μl of each diluted compound was removed to a new plate and diluted to 200 μl (final volume) with 160 μl pNPP solution and 20 μl PTP1B solution. Reactions were thus started immediately and were stopped after 60 minutes by addition of 100 μl 0.1N NaOH. The OD⁴⁰⁵ was subsequently measured. Two wells on each plate contained DMSO controls and two wells contained sodium orthovanadate (2 mM) which inhibits PTP1B-catalyzed hydrolysis of pNPP completely. Data were corrected for background absorbance by the subtraction of the optical densities from a no-enzyme control plate and were expressed as percent inhibition relative to the average of the vanadate controls measured on the same microtiter plate. The activity of the other PTPs was determined in a similar fashion except that the concentration of pNPP was varied according to the Km values for individual enzymes (0.6 mM for TCPTP and 6.25 mM for each of SHP-2 and LAR) and the buffer used for TCPTP was 25 mM Tris-HCl pH 7.2.

Cell-based Analysis of Compound Activity

The effects of compounds on the phosphorylation status of the insulin receptor was measured using L6 muscle cells expressing the receptor endogenously. L6 myocytes were cultured in α-MEM with 10% foetal bovine serum and antibiotics. Cells were differentiated into myotubes in 24-well plates by culturing for 10 days in medium containing 2% serum. The medium was refreshed on alternate days and 0.24 mg/ml cytidine was included from day 7 to stop any remaining cycling cells. Cells were starved of serum overnight prior to use. Cells were pretreated with compound at approximately five times the IC₅₀ for inhibition of PTP 1 B for 30 minutes, prior to being stimulated with insulin (25 nM) for five minutes. Cells were lysed in buffer comprising 25 mM Tris-HCl pH 7.4, 150 mM NaCl, 1 mM EDTA, 1 mM EGTA, 1% Nonidet-40, 0.25% sodium deoxycholate, 1 mM sodium orthovanadate, 10 mM β-glycerophosphate, 5 mM sodium pyrophosphate and protease inhibitors. Cleared lysates were stored at −70° C. until used.

Insulin receptor phosphorylation was assessed using a lanthanide-based fluorescent assay (DELFIA). An anti-insulin receptor antibody was captured on the wells of a 96-well plate using an anti-rabbit IgG antibody. After incubation with lysates containing between 100-250 μg protein which was consistent for all wells in a single experiment, phosphate on the receptor was detected with a biotinylated anti-phosphotyrosine antibody (PY99B from Santa Cruz) and europium-labelled streptavidin.

Results

IC₅₀ values were determined for all compounds against each of four PTPs (PTP1B, SHP-2, LAR and TCPTP). Compounds were active across a wide range of concentrations from 100 nM to 50 μM.

Representative compounds from across the series were analysed in cell-based assays in which effects on insulin-stimulated auto-phosphorylation of the insulin receptor was determined. Compounds caused statistically-significant increases in receptor phosphorylation by between 20% to 70%.

What is claimed is:

1. A compound of the formula I 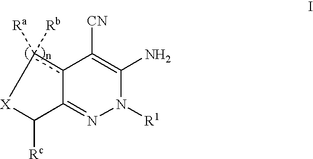
wherein
n is an integer of 1 or 2;
R¹ is
(i) phenyl or naphthyl, each optionally substituted with up to 5 substituents independently selected from the group consisting of:
(a) a straight or branched C₁-C₆ alkyl;
(b) a C₃-C₆ cycloalkyl or a C₆-C₁₀ aryl;
(c) —CO—O-(C₁-C₆ alkyl) wherein the alkyl group is straight or branched;
(d) a halogen selected from the group consisting of fluoro, chloro, bromo, and iodo;
(e) a straight or branched C₁-C₆ alkoxy;
(f) nitro;
(g) CF₃;
(h) —O-(C₁-C₆ alkyl)-phenyl wherein the alkyl group is straight or branched;
(i) a heteroaryl having 5 or 6 ring atoms, wherein 1 or 2 of the ring atoms are optionally O, N, or S, optionally substituted with one or more methyl or ethyl;
(j) a heterocycloalkyl having 5 or 6 ring atoms, wherein 1 or 2 of the ring atoms are optionally O, N, or S;
(k) —N(R²)—CO—R³, where R² is hydrogen or a straight or branched C₁-C₆ alkyl, and R³ is a straight or branched C₁-C₆ alkyl; and
(l) —N(R⁴)(R⁵), where each of R⁴ and R⁵ independently is a straight or branched C₁-C₆ alkyl;
(ii) phenyl that is fused with a cyclohexyl group or naphthyl that is fused with a cyclohexyl group, wherein 1 or 2 of the carbon atoms are optionally substituted with O, N, or S, and wherein said cyclohexyl group is optionally substituted with 1 or 2 substituents independently selected from the group consisting of halogen, CF₃, a straight or branched C₁-C₆ alkyl, a C₃-C₆ cycloalkyl, and a C₆-C₁₀ aryl; or
(iii) 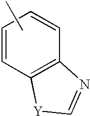
wherein Y is O, S, or N—R^{X} wherein R^{X} is a straight or branched C₁-C₆ alkyl;
X is
(i) an oxygen atom, a sulfur atom, or a methylene group; 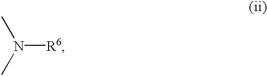
wherein R⁶ is
(a) benzyl;
(b) —CO-(C₁-C₆ alkyl) wherein the alkyl group is straight or branched;
(c) —CO—A, wherein A is phenyl, naphthyl, or a heteroaryl; said heteroaryl group having 5 to 10 ring atoms wherein at least one of said ring atoms is O, N, or S;
(d) —CO—O—R^{y}, wherein R^{y} is hydrogen or a straight or branched C₁-C₆ alkyl;
(e) —CO—O-(C₁-C₆ alkyl)-A wherein A is as defined above; or
(f) —SO₂—A, wherein A is as defined above; 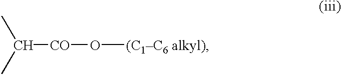
wherein the alkyl group is straight or branched; or 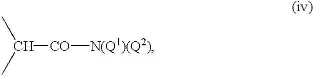
wherein each of Q¹ and Q² independently is phenyl, naphthyl, or a heteroaryl; said heteroaryl group having 5 to 10 ring atoms wherein at least one of said ring atoms is O, N, or S;
each of R^{a} and R^{b} is independently hydrogen or C₁-C₆ alkyl, or R^{a} and R^{b} together form a carbonyl group; and
R^{c} is hydrogen, a straight or branched C₁-C₆ alkyl, or carbonyl;
with the proviso that the compound 6H-pyrano[3,4-c]pyridazine-4-carbonitrile, 3-amino-2,8-dihydro-6,6-dimethyl-2-phenyl is excluded;
or a pharmaceutically and pharmacologically acceptable salt or hydrate thereof.

2. The compound of claim 1, said compound having formula Ia: 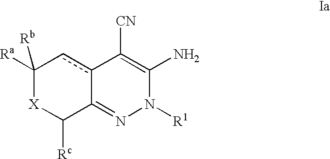
wherein X, R¹, R^{a}, R^{b}, and R^{c} are as defined in claim 1.

3. The compound of claim 1, said compound having formula Ia′: 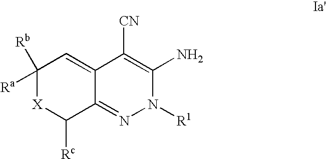
wherein X, R¹, R^{a}, R^{b}, and R^{c} are as defined in claim 1.

4. The compound of claim 1, said compound having formula Ib: 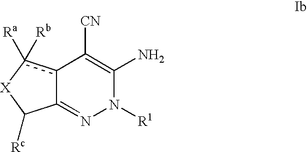
wherein X, R¹, R^{a}, R^{b}, and R^{c} are as defined in claim 1.

5. The compound of claim 1, wherein R¹ is phenyl or naphthyl, each optionally and independently substituted with 1, 2, 3, 4, or 5 substituents selected from the group consisting of methyl; ethyl; straight, branched or cyclic propyl, butyl, pentyl, or hexyl; —CO—O—(CH₂)ₙ—CH₃ wherein n is an integer 0, 1, 2, 3, 4, or 5; methoxy, ethoxy, propoxy, butyloxy, pentyloxy, or hexyloxy; and —O—(CH₂)ₙ-phenyl where n is an integer 0, 1, 2, 3, 4, 5, or 6.

6. The compound of claim 1, wherein R¹ is phenyl, optionally substituted with a straight or branched C₁-C₄ alkyl, a straight or branched C₁-C₆ alkoxy, nitro, CF₃, fluoro, chloro, bromo, cyclohexyl, heterocyclohexyl, phenyl, —CO—O-(C₁-C₄ alkyl), or —N(R²)—CO—R³, where R² is hydrogen, methyl, or ethyl, and R³ is a straight or branched C₁-C₄ alkyl.

7. The compound of claim 1, wherein R¹ is phenyl, optionally substituted with methyl, ethyl, n-propyl, isopropyl, methoxy, ethoxy, propoxy, CF₃, morpholino, fluoro, chloro, phenyl, —CO—O—CH₃, —CO—O—CH₂CH₃, —NH—CO—CH₃, or —NH—CO—CH₂CH₃.

8. The compound of claim 1, wherein R¹ is 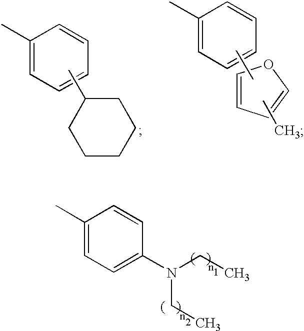
wherein each of n₁ and n₂ independently is an integer 0, 1, 2, 3, 4, or 5; 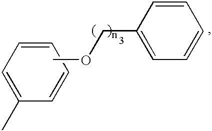
wherein n₃ is an integer 0, 1, 2, 3, 4, or 5; or 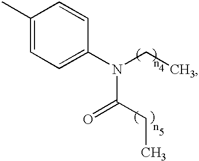
wherein each of n₄ and n₅ independently is an integer 0, 1, 2, 3, 4, or 5.

9. The compound of claim 1, wherein R¹ is

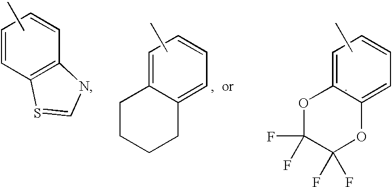

10. The compound of claim 1, wherein X is an oxygen atom; a sulfur atom; a methylene group; 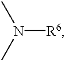
wherein R⁶ is benzyl, —CO-(C₁-C₄ alkyl), —CO—O-(C₁-C₄ alkyl); or

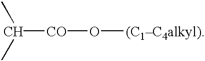

11. The compound of claim 1, wherein X is an oxygen atom.

12. The compound of claim 1, wherein each of R^{a} and R^{b} is independently hydrogen, methyl, or ethyl.

13. The compound of claim 1, wherein R^{c} is hydrogen, methyl, or ethyl.

14. The compound of claim 3, wherein
R¹ is phenyl, optionally substituted with a straight or branched C₁-C₄ alkyl, a straight or branched C₁-C₆ alkoxy, nitro, CF₃, fluoro, chloro, bromo, cyclohexyl, heterocyclohexyl, phenyl, —CO—O-(C₁-C₄ alkyl), or —N(R²)—CO—R³, where R² is hydrogen, methyl, or ethyl, and R³ is a straight or branched C₁-C₄ alkyl;
X is an oxygen atom; a sulfur atom; a methylene group; 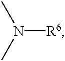
wherein R⁶ is benzyl, —CO-(C₁-C₄ alkyl), or —CO—O-(C₁-C₄ alkyl); or 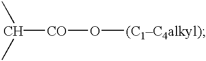
and
each of R^{a}, R^{b}, and R^{c} independently is hydrogen, methyl, or ethyl.

15. The compound of claim 3, wherein
R¹ is 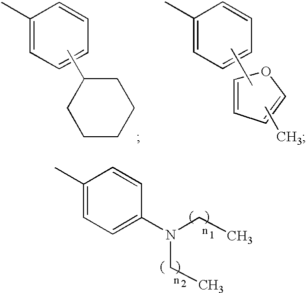
wherein each of n₁ and n₂ independently is an integer 0, 1, 2, 3, 4, or 5; 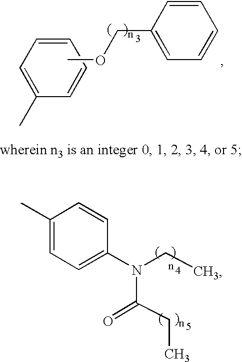
wherein n₃ is an integer 0, 1, 2, 3, 4, or 5;
wherein each of n₄ and n₅ independently is an integer 0, 1, 2, 3, 4, or 5; 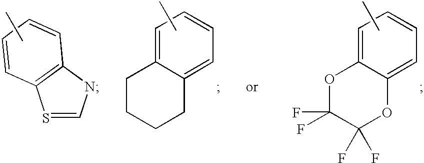
X is an oxygen atom; a sulfur atom; a methylene group; 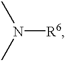
wherein R⁶ is benzyl, —CO-(C₁-C₄ alkyl), or —CO—O-(C₁-C₄ alkyl); or 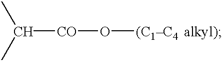
and
each of R^{a}, R^{b}, and R^{c} independently is hydrogen, methyl, or ethyl.

16. The compound of claim 1, said compound being
3-amino-2-phenyl-2,8-dihydro-6H-pyrano[3,4-c]pyridazine-4-carbonitrile;
3-amino-2-phenyl-2,6,7,8-tetrahydro-4-cinnolinecarbonitrile;
3-amino-2-phenyl-6,7-dihydro-2H-cyclopenta[c]pyridazine-4-carbonitrile;
3-amino-7-benzyl-2-phenyl-2,6,7,8-tetrahydropyrido[3,4-c]pyridazine-4-carbonitrile;
7-acetyl-3-amino-2-phenyl-2,6,7,8-tetrahydropyrido[3,4-c]pyridazine-4-carbonitrile;
3-amino-2-(2-fluorophenyl)-2,8-dihydro-6H-pyrano[3,4-c]pyridazine-4-carbonitrile;
3-amino-2-(4-methylphenyl)-2,8-dihydro-6H-pyrano[3,4-c]pyridazine-4-carbonitrile;
3-amino-2-[3-(trifluoromethyl)phenyl]-2,8-dihydro-6H-pyrano[3,4-c]pyridazine-4-carbonitrile;
3-amino-2-phenyl-2,8-dihydro-6H-thiopyrano[3,4-c]pyridazine-4-carbonitrile;
3-amino -2-(3-methoxyphenyl)-2,8-dihydro-6H-pyrano[3,4-c]pyridazine-4-carbonitrile;
3-amino-2-(3- methoxyphenyl)-2,8-dihydro-6H-pyrano[3,4-c]pyridazine-4-carbonitrile;
ethyl 3-amino -4-cyano-2-phenyl-2,6,7,8-tetrahydro-7-cinnolinecarboxylate;
3-amino-2-(3,4-dichlorophenyl)-2,8-dihydro-6H-pyrano[3,4-c]pyridazine-4-carbonitrile;
7-benzoyl-3-amino-2-phenyl-2,6,7,8-tetrahydropyrido[3,4-c]pyridazine-4-carbonitrile;
3-amino-2-(4-methoxyphenyl)-2,8-dihydro-6H-pyrano[3,4-c]pyridazine-4-carbonitrile;
3-amino-2-(4-isopropylphenyl)-2,8-dihydro-6H-pyrano[3,4-c]pyridazine-4-carbonitrile;
3-amino-2-[4-(4-morpholinyl)phenyl]-2,8-dihydro-6H-pyrano[3,4-c]pyridazine-4-carbonitrile;
7-acetyl-3-amino-2-(2,6-dimethylphenyl)-2,6,7,8-tetrahydropyrido[3,4-c]pyridazine-4-carbonitrile;
ethyl 3-(3-amino-4-cyano-6H-pyrano[3,4-c]pyridazin-2(8H)-yl)benzoate;
ethyl 3-amino-4-cyano-2-phenyl-2,8-dihydropyrido[3,4-c]pyridazine-7(6H)-carboxylate;
3-amino-2-(2-chloro-6-fluorophenyl)-2,8-dihydro-6H-pyrano[3,4-c]pyridazine-4-carbonitrile;
3-amino-2-(2-bromo-5-trifluoromethylphenyl)-2,8-dihydro-6H-pyrano[3,4-c]pyridazine-4-carbonitrile;
3-amino-2-(1,3-benzothiazol-6-yl)-2,8-dihydro-6H-pyrano[3,4-c]pyridazine-4-carbonitrile;
N-[4-(3-amino-4-cyano-6H-pyrano[3,4-c]pyridazin-2(8H)-yl)phenyl]-N-methylacetamide;
3-amino-2-[4-(dimethylamino)phenyl]-2,8-dihydro-6H-pyrano[3,4-c]pyridazine-4-carbonitrile;
3-amino-2-(4-chloro-2-methylphenyl)-2,8-dihydro-6H-pyrano[3,4-c]pyridazine-4-carbonitrile;
3-amino-2-(2-chloro-6-methylphenyl)-2,8-dihydro-6H-pyrano[3,4-c]pyridazine-4-carbonitrile;
3-amino-2-(2-methoxyphenyl)-2,8-dihydro-6H-pyrano[3 ,4-c]pyridazine-4-carbonitrile;
3-amino-2-mesityl-2,8-dihydro-6H-pyrano[3,4-c]pyridazine-4-carbonitrile;
3-amino-2-(2-isopropylphenyl)-2,8-dihydro-6H-pyrano[3,4-c]pyridazine-4-carbonitrile;
3-amino-2-(2,6-dichlorophenyl)-2,8-dihydro-6H-pyrano[3,4-c]pyridazine-4-carbonitrile;
3-amino-4-cyano-2-phenyl-2,8-dihydropyrido[3,4-c]pyridazine-7(6H)-carboxylate;
3-amino-2-(2-methoxy-6-methylphenyl)-2,8-dihydro-6H-pyrano[3,4-c]pyridazine-4-carbonitrile;
3-amino-2-(2-methyl-4-nitrophenyl)-2,8-dihydro-6H-pyrano[3,4-c]pyridazine-4-carbonitrile;
3-amino-2-(3,5-dimethoxyphenyl)-2,8-dihydro-6H-pyrano[3,4-c]pyridazine-4-carbonitrile;
3-amino-2-(2-(1-methylpropyl)phenyl)-2,8-dihydro-6H-pyrano[3,4-c]pyridazine-4-carbonitrile;
3-amino-2-(2-chloro-5-methoxyphenyl)-2,8-dihydro-6H-pyrano[3,4-c]pyridazine-4-carbonitrile;
3-amino-2-(2-ter-butylphenyl)-2,8-dihydro-6H-pyrano[3,4-c]pyridazine-4-carbonitrile;
3-amino-2-(2-propylphenyl)-2,8-dihydro-6H-pyrano[3,4-c]pyridazine-4-carbonitrile;
3-amino-2-[2-(4-morpholinyl)phenyl]-2,8-dihydro-6H-pyrano[3,4-c]pyridazine-4-carbonitrile;
3-amino-2-(5,6,7,8-tetrahydro-1-naphthalenyl)-2,8-dihydro-6H-pyrano[3,4-c]pyridazine-4-carbonitrile;
3-amino-2-[1,1′-biphenyl]-2-yl-2,8-dihydro-6H-pyrano[3,4-c]pyridazine-4-carbonitrile;
3-amino-2-[2-(5-methyl-2-furyl)phenyl]-2,8-dihydro-6H-pyrano[3,4-c]pyridazine-4-carbonitrile;
3-amino-2-(2,2,3,3-tetrafluoro-2,3-dihydro-1,4-benzodioxin-5-yl)-2,8-dihydro-6H-pyrano[3,4-c]pyridazine-4-carbonitrile;
3-amino-2-(2,2,3,3-tetrafluoro-2,3-dihydro-1,4-benzodioxin-5-yl)-2,8-dihydro-6H-pyrano[3,4-c]pyridazine-4-carbonitrile; or
3-amino-2-phenyl-2,5,6,8-tetrahydro-3H-pyrano[3,4-c]pyridazine-4-carbonitrile.

17. A hydrochloride salt or tosylate salt of a compound of claim 1.

18. A hydrochloride salt of a compound of claim 1.

19. A compound of claim 18, said compound being
3-amino-2-(2-fluorophenyl)-2,8-dihydro-6H-pyrano[3,4-c]pyridazine-4-carbonitrile hydrochloride (compound 9);
3-amino-2-(4-methylphenyl)-2,8-dihydro-6H-pyrano[3,4-c]pyridazine-4-carbonitrile hydrochloride (compound 10);
3-amino-2-[3-(trifluoromethyl)phenyl]-2,8-dihydro-6H-pyrano[3,4-c]pyridazine-4-carbonitrile hydrochloride (compound 11);
3-amino-2-phenyl-2,8-dihydro-6H-thiopyrano[3,4-c]pyridazine-4-carbonitrile hydrochloride (compound 12);
3-amino-2-(3-methoxyphenyl)-2,8-dihydro-6H-pyrano[3,4-c]pyridazine-4-carbonitrile hydrochloride (compound 13);
3-amino-2-(3-methoxyphenyl)-2,8-dihydro-6H-pyrano[3,4-c]pyridazine-4-carbonitrile hydrochloride (compound 14);
ethyl 3-amino-4-cyano-2-phenyl-2,6,7,8-tetrahydro-7-cinnolinecarboxylate hydrochloride (compound 15);
3-amino-2-(3,4-dichlorophenyl)-2,8-dihydro-6H-pyrano[3,4-c]pyridazine-4-carbonitrile hydrochloride (compound 16);
3-amino-2-(4-methoxyphenyl)-2,8-dihydro-6H-pyrano[3,4-c]pyridazine-4-carbonitrile hydrochloride (compound 18);
3-amino-2-(4-isopropylphenyl)-2,8-dihydro-6H-pyrano[3,4-c]pyridazine-4-carbonitrile hydrochloride (compound 19);
3-amino-2-[4-(4-morpholinyl)phenyl]-2,8-dihydro-6H-pyrano[3,4-c]pyridazine-4-carbonitrile hydrochloride (compound 20);
7-acetyl-3-amino-2-(2,6-dimethylphenyl)-2,6,7,8-tetrahydropyrido[3,4-c]pyridazine-4-carbonitrile hydrochloride (compound 21);
ethyl 3-(3-amino-4-cyano-6H-pyrano[3,4-c]pyridazin-2(8H)-yl)benzoate hydrochloride (compound 22);
ethyl 3-amino-4-cyano-2-phenyl-2,8-dihydropyrido[3,4-c]pyridazine-7(6H)-carboxylate hydrochloride (compound 23);
3-amino-2-(2-chloro-6-fluorophenyl)-2,8-dihydro-6H-pyrano[3,4-c]pyridazine-4-carbonitrile hydrochloride (compound 24);
3-amino-2-(2-bromo-5-trifluoromethylphenyl)-2,8-dihydro-6H-pyrano[3,4-c]pyridazine-4-carbonitrile hydrochloride (compound 25);
3-amino-2-(1,3-benzothiazol-6-yl)-2,8-dihydro-6H-pyrano[3,4-c]pyridazine-4-carbonitrile hydrochloride (compound 26);
N-[4-(3-amino-4-cyano-6H-pyrano[3,4-c]pyridazin-2(8H)-yl)phenyl]-N-methylacetamide hydrochloride (compound 27);
3-amino-2-[4-(dimethylamino)phenyl]-2,8-dihydro-6H-pyrano[3,4-c]pyridazine-4-carbonitrile hydrochloride (compound 28);
3-amino-2-(4-chloro-2-methylphenyl)-2,8-dihydro-6H-pyrano[3,4-c]pyridazine-4-carbonitrile hydrochloride (compound 29);
3-amino-2-(2-chloro-6-methylphenyl)-2,8-dihydro-6H-pyrano[3,4-c]pyridazine-4-carbonitrile hydrochloride (compound 30);
3-amino-2-(2-methoxyphenyl)-2,8-dihydro-6H-pyrano[3,4-c]pyridazine-4-carbonitrile hydrochloride (compound 31);
3-amino-2-mesityl-2,8-dihydro-6H-pyrano[3,4-c]pyridazine-4-carbonitrile hydrochloride (compound 32);
3-amino-2-(2-isopropylphenyl)-2,8-dihydro-6H-pyrano[3,4-c]pyridazine-4-carbonitrile hydrochloride (compound 33);
3-amino-2-(2,6-dichlorophenyl)-2,8-dihydro-6H-pyrano[3,4-c]pyridazine-4-carbonitrile hydrochloride (compound 34);
3-amino-2-(2-methyl-4-nitrophenyl)-2,8-dihydro-6H-pyrano[3,4-c]pyridazine-4-carbonitrile hydrochloride (HCl salt of compound 35);
3-amino-4-cyano-2-phenyl-2,8-dihydropyrido[3,4-c]pyridazine-7(6H)-carboxylate hydrochloride (compound 36);
3-amino-2-(2-methoxy-6-methylphenyl)-2,8-dihydro-6H-pyrano[3,4-c]pyridazine-4-carbonitrile hydrochloride (compound 37);
3-amino-2-(3,5-dimethoxyphenyl)-2,8-dihydro-6H-pyrano[3,4-c]pyridazine-4-carbonitrile hydrochloride (compound 38);
3-amino-2-(2-(1-methylpropyl)phenyl)-2,8-dihydro-6H-pyrano[3,4-c]pyridazine-4-carbonitrile hydrochloride (compound 39); 3-amino-2-(2-chloro-5-methoxyphenyl)-2,8-dihydro-6H-pyrano[3,4-c]pyridazine-4-carbonitrile hydrochloride (compound 40);
3-amino-2-(2-ter-butylphenyl)-2,8-dihydro-6H-pyrano[3,4-c]pyridazine-4-carbonitrile hydrochloride (compound 41);
3-amino-2-(2-propylphenyl)-2,8-dihydro-6H-pyrano[3,4-c]pyridazine-4-carbonitrile hydrochloride (compound 42); or
3-amino-2- [2-(4-morpholinyl)phenyl]-2,8-dihydro-6H-pyrano[3,4-c]pyridazine-4-carbonitrile hydrochloride (compound 43).

20. A pharmaceutical composition comprising a compound of claim 1 and a pharmacologically and pharmaceutically acceptable carrier.

21. A pharmaceutical composition comprising a compound of claim 15 and a pharmacologically and pharmaceutically acceptable carrier.

22. A pharmaceutical composition comprising a compound of claim 16 and a pharmacologically and pharmaceutically acceptable carrier.

23. A pharmaceutical composition comprising a compound of claim 19 and a pharmacologically and pharmaceutically acceptable carrier.

24. A method for treating type 2 diabetes mellitus, comprising administering to a subject in need thereof an effective amount of a compound of formula I 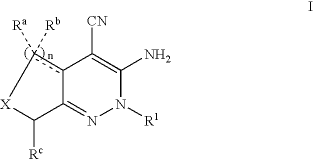
wherein
n is an integer of 1 or 2;
R¹ is
(i) phenyl or naphthyl, each optionally substituted with up to 5 substituents independently selected from the group consisting of:
(a) a straight or branched C₁-C₆ alkyl;
(b) a C₃-C₆ cycloalkyl or a C₆-C₁₀ aryl;
(c) —CO—O-(C₁-C₆ alkyl) wherein the alkyl group is straight or branched;
(d) a halogen selected from the group consisting of fluoro, chloro, bromo, and iodo;
(e) a straight or branched C₁-C₆ alkoxy;
(f) nitro;
(g) CF₃;
(h) —O-(C₁-C₆ alkyl)-phenyl wherein the alkyl group is straight or branched;
(i) a heteroaryl having 5 or 6 ring atoms, wherein 1 or 2 of the ring atoms are optionally O, N, or S, optionally substituted with one or more methyl or ethyl;
(j) a heterocycloalkyl having 5 or 6 ring atoms, wherein 1 or 2 of the ring atoms are optionally O, N, or S;
(k) —N(R²)—CO—R³, where R² is hydrogen or a straight or branched C₁-C₆ alkyl, and R³ is a straight or branched C₁-C₆ alkyl; and
(l) —N(R⁴)(R⁵), where each of R⁴ and R⁵ independently is a straight or branched C₁-C₆ alkyl;
(ii) phenyl that is fused with a cyclohexyl group or naphthyl that is fused with a cyclohexyl group, wherein 1 or 2 of the carbon atoms are optionally substituted with O, N, or S, and wherein said cyclohexyl group is optionally substituted with 1 or 2 substituents independently selected from the group consisting of halogen, CF₃, a straight or branched C₁-C₆ alkyl, a C₃-C₆ cycloalkyl, and a C₆-C₁₀ aryl; or 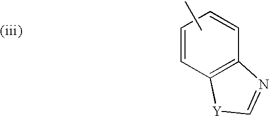
wherein Y is O, S, or N—R^{X} wherein R^{X} is a straight or branched C₁-C₆ alkyl;
X is
(i) an oxygen atom, a sulfur atom, or a methylene group; 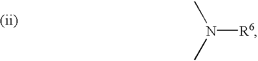
wherein R⁶ is
(a) benzyl;
(b) —CO-(C₁-C₆ alkyl) wherein the alkyl group is straight or branched;
(c) —CO—A, wherein A is phenyl, naphthyl, or a heteroaryl; said heteroaryl group having 5 to 10 ring atoms wherein at least one of said ring atoms is O, N, or S;
(d) —CO—O—R^{y}, wherein R^{y} is hydrogen or a straight or branched C₁-C₆ alkyl;
(e) —CO—O-(C₁-C₆ alkyl)-A wherein A is as defined above; or
(f) —SO₂—A, wherein A is as defined above; 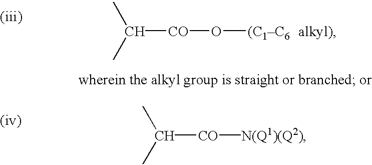
wherein the alkyl group is straight or branched; or
wherein each of Q¹ and Q² is independently phenyl, naphthyl, or a heteroaryl; said heteroaryl group having 5 to 10 ring atoms wherein at least one of said ring atoms is O, N, or S;
each of R^{a} and R^{b} is independently hydrogen or C₁-C₆ alkyl, or R^{a} and R^{b} together form a carbonyl group; and
R^{c} is hydrogen, a straight or branched C₁-C₆ alkyl, or carbonyl;
or a pharmaceutically and pharmacologically acceptable salt or hydrate thereof.

25. A method for treating type 2 diabetes mellitus, comprising administering to a subject in need thereof an effective amount of a compound of claim 2.

26. A method for treating type 2 diabetes mellitus, comprising administering to a subject in need thereof an effective amount of a compound of claim 3.

27. A method for treating type 2 diabetes mellitus, comprising administering to a subject in need thereof an effective amount of a compound of claim 16.

28. A method for treating type 2 diabetes mellitus, comprising administering to a subject in need thereof an effective amount of a compound of claim 19.
